(19) United States
(12) Patent Application Publication
(10) Pub. No.: US 2008/0020393 A1
(43) Pub. Date: Jan. 24, 2008

(54) IRON REGULATING PROTEIN-2 (IRP-2) AS A DIAGNOSTIC FOR NEURODEGENERATIVE DISEASE

(75) Inventors: Wolff M. Kirsch, Redlands, CA (US); Anton Lennart, Loma Linda, CA (US); Wayne J. Kelln, Loma Linda, CA (US); Dae-Kyung Kang, Rockville, MD (US); Rodney L. Levine, Rockville, MD (US); Tracey A. Rouault, North Bethesda, MD (US)

Correspondence Address: KNOBBE MARTENS OLSON & BEAR LLP, 2040 MAIN STREET, FOURTEENTH FLOOR, IRVINE, CA, 92614, US

(73) Assignee: LOMA LINDA UNIVERSITY MEDICAL CENTER, Loma Linda, CA (US)

(21) Appl. No.: 11/769,503

(22) Filed: Jun. 27, 2007

Related U.S. Application Data
(63) Continuation of application No. 11/174,166, filed on Jul. 1, 2005, now abandoned.
Application No. 11/174,166 is a continuation of application No. 09/924,396, filed on Aug. 6, 2001, now abandoned.
(60) Provisional application No. 60/222,863, filed on Aug. 4, 2000.

(51) Int. Cl. C12Q 1/68; G01N 33/567

(52) U.S. Cl. 435/6; 435/7.21

(57) ABSTRACT

The present invention relates to the discovery of markers for neurodegenerative disease. More particularly, it was discovered that forms of IRP-2 protein that are unable to undergo oxidation at critical cysteine residues are diagnostic for neurodegenerative disease including, but not limited to Alzheimer's disease. Embodiments include nucleic acids that encode mutant IRP-2 proteins and fragments thereof, mutant IRP-2 proteins and fragments thereof, antibodies directed to epitopes present on mutant IRP-2 proteins and fragments thereof, methods of making these nucleic acids and polypeptides, as well as, approaches to diagnose neurodegenerative disease in animals, such as humans at risk of contracting Alzheimer's disease.

### RELATED APPLICATION

The present application is a continuation of U.S. Ser. No. 11/174,166, filed Jul. 1, 2005, which is a continuation of U.S. Ser. No. 09/924,396, filed Aug. 6, 2001, which claims the benefit under 35 U.S.C. §119(e) of U.S. Provisional Application No. 60/222,863, filed Aug. 4, 2000, all of which are incorporated by reference in their entirety.

This invention was made in part with Government support under Grant No. ______, awarded by the National Institutes of Health. The Government may have certain rights in this invention.

### FIELD OF THE INVENTION

The present invention relates to the discovery of antibodies which can be used to diagnose and identify patients with neurodegenerative disease (Alzheimer's disease). More particularly, the antibodies recognize a specific region of the iron regulating protein 2 (IRP-2).

### BACKGROUND OF THE INVENTION

Neurodegenerative disease plagues several million people world-wide. Alzheimer's disease (AD), for example, is the fourth most common cause of death in the United States after heart disease, cancer, and stroke. It presently afflicts more than 4 million people in the United States alone and this number is expected to double during the next 40 years as the population ages. Apart from advanced age and Downs syndrome, the only consistent risk factor for the development of neurodegenerative disease has been the presence of a positive family history. Currently, investigators are performing genetic linkage analysis to identify diseased genes that contribute to neurodegenerative disease; however, the understanding of the biochemical mechanisms that underlie these maladies remains in its infancy.

Recently, however, there has been an increase in suspicion that a disturbance in brain iron regulation contributes to some forms of neurodegenerative disease, for example, AD (Gerlach et al., J. Neurochem. 63:793-807 (1994)). In the brain, iron metabolism is tightly controlled. An excess of iron results in toxicity and too little impairs metabolism. All tissues regulate iron uptake through the action of iron regulatory protein 1 (IRP-1) and iron regulatory protein 2 (IRP-2). Recent findings reveal that that these iron regulatory proteins, particularly IRP-2, are involved in the impaired iron homeostasis that is observed in patients suffering from Alzheimer's disease. (Smith et al., Brain Research, 788:232-236 (1998)).

In iron deficient cells, for example, an increase in IRP-2 levels is observed. As a result of this increase, IRP-2 binds to the 3′ prime untranslated region of the mRNA for transferrin receptor, which is a protein that facilitates iron uptake. Additionally, IRP-2 prevents binding to the 5′ cap structure of HnRNA encoding ferritin blocking binding and subsequent translation. In essence, iron uptake is facilitated by the presence of high levels of IRP-2. On the other hand, if cells are provided an excess of iron, IRP-2 is rapidly degraded and iron uptake is immediately reduced. Thus, the body achieves iron homeostasis by regulating the degradation of IRP-2. (Van Buskirk et al., Proc. Natl. Acad. Sci., 81:722-725 (1984)). A greater understanding of the induction of IRP-2 degradation is needed.

Though there are a number of reports in the literature on the quantitation of brain iron by MRI (Scheffler et al., Magn Reson Med., 42(5):829-36 (1999); Vymazal et al., J Neurol Sci., 134 Suppl:19-26 (1995); Quast et al., Magn Reson Imaging, 11 (4):465-71 (1993)), no universally accepted methods or standards and no calibrated or verified data on humans exist. Serial, longitudinal studies looking for differences in rates of change for example, of temporal lobe and hippocampal volume, have proven to be more powerful diagnostic aids than isolated measurements. Temporal lobe atrophy has been followed serially in AD patients with MR imaging techniques. The ability to assess quantitatively and sequentially regional brain iron provides potential utility in both diagnosis and monitoring of prospective treatments of individuals with neurodegenerative disorders.

Iron has numerous effects on MR images in its paramagnetic form. Effects include signal changes in magnitude and phase images in T2* weighted gradient echo images, signal changes in T2 weighted and diffusion weighted spin echo images, and signal increases in T1 weighted images. In gray matter where iron content is high (such as in the central sulcus), iron behaves as a T1 reducing contrast agent.

One major source of brain iron is the ferric form of ferritin which plays a major role in storage and utilization of iron in the brain. Each ferritin molecule consists of different ratios of H (heavy) and L (light) chain subunits which are coded on different chromosomes, and play different roles in the function of the ferritin molecule. The H-rich ferritin is efficient at iron sequestration and is predominant in organs with high iron utilization and little iron storage while L-rich ferritin is efficient at iron nucleation and is associated with iron storage. In the brain, various cell types contain ferritin isoforms that are consistent with their functional roles. Ferritin has unique magnetic properties and is believed to be the major source of iron-induced changes in MR tissue relaxation times. The amount of ferritin is ten times the amount of transferrin in the brain with each ferritin molecule having the ability to sequester up to 4500 iron atoms. Ferritin is stored in oligodendrocytes, astrocytes and myelin in microglia. Macrophages can convert ferritin to hemosiderin, another potent paramagnetic substance that generates signal changes in T2* weighted MRI images. Despite this general knowledge, the MRI properties of ferritin are not well understood. The expected field dependence of R2 is the square of the static field. To the contrary, all evidence points to a linear change in R2 with field strength. Further, relaxation rates are generally found to be too high to be explained by simple paramagnetisim. A recent paper quotes relaxivities of ferritin for R1 of 2.19±0.05/s/mg Fe/g, a value consistent with other measurements (Gossuin et al., Magn Reson Med, 43(2):237-43 (2000)).

The second major source of brain iron is free iron. Other sources of trapped iron exist but their concentrations are small. In agreement with the phase measurements, R2 or R2′ data and other measures of brain iron, the basal ganglia contain more stainable iron than the cerebral hemispheres and white matter. From assays of brain iron post-mortem, iron levels are 2 μg/gm in the red nucleus for elderly individuals while normal levels in the globus pallidus are about 0.25 μg/gm tissue. Other observations include increased iron stores in the hippocampus in Alzheimer's disease and Parkinson's disease, increased ferritin in grey matter upon aging, and unchanged levels of astrocyte iron.

High field results on animals (Fenzi et al., J Magn Reson Imaging, 13(3):392-6 (2001)) and humans (Bonkovsky et al., Radiology, 212(1):227-34 (1999); Bartzokis et al., Cell Mol Biol (Noisy-le-grand), 46(4):821-33 (2000)) have attempted to quantify brain iron. Although the trend clearly demonstrates an increase in R2 as iron content increases, the predictability of the results is difficult. For example, Fenzi shows that the slope for R2 is 10 to 30/s/(mg/gm Fe) on a phantom with an R2 of 40/s when there is no iron. However, in vivo, a single T2 of 150/s can correspond to a range of 1.5 to 3.5 mg Fe/gm wet weight, far too broad to be of clinical value. Similarly, Bonkovsky's data show that a single signal intensity measurement corresponds to a range of 2/mg/gm dry liver for low concentrations and 5/mg/gm dry liver for concentrations above 1/mg/g dry liver.

Ordidge and his group (Mizkeil et al., Magn Reson Imaging, 15(10):1113-9 (1997) demonstrated that key information lies in R2′ not R2. A problem with R2 results because of other effects that can change T2 and confound information about local iron. Signal for long echoes despite the local increase in iron in the substantia nigra are anomalously recovered with R2′ continuing to increase with an increase in iron content. A method was developed by Ordidge et al. to measure R2′ despite the presence of background field variations that dephase the signal and otherwise yield a falsely high value for R2′. The local field in the slice select direction was compensated by repeating the scan multiple times using different slice select gradients.

Gelman et al. measured both R2 and R2′ effects and found (Radiology, 213(1):135-40 (1999) that the slope of R2 is 60/s/mg wet wt with an intercept 12.7 or T2 of about 80 ms, the slope of R2′ is 50/s/mg wet wt with an intercept 2.7 (one might postulate that this non-zero intercept may represent the heme iron contribution) and as an example, the R2′ of globus pallidus is 12/sec. In fact, a number of papers have demonstrated the T2 and T2* effects of iron in the basal ganglia and liver. Further, diffusion mechanisms have been used to describe the signal loss with iron. More recently, theories involving spin dephasing in the static or slow diffusion regime and in the fast diffusion regime. _This unique feature has been considered when evaluating parallel fibres (Hajnal et al., J Comput Assist Tomogr., 16(4):506-13 (1992)) and to measure oxygen content in the brain (An and Lin, J Cereb Blood Flow Metab., 20(8):1225-36 (2000)).

T2* measurements and R2′ quantification are now considered optimum for brain iron measurements. Gillet et al. (J Neurol Sci., 168(1):21-7 (1999)) uses a 3D gradient echo structure with a TE=9 ms at a field strength of 11.7 T, almost exactly the equivalent of what we use at 1.5 T for the best phase contrast images. Iron is seen in basal forebrain cholinergic structures such as the basalis of Meynert in a well established mouse model that has the neuropathological hallmarks, including senile plaques and neurofibrillary tangles, of AD, while a high iron content is observed in the globus pallidus in AD.

Forgetful individuals most likely to develop AD have a condition known as mild cognitive disorder or mild cognitive impairment syndrome (MCI) previous to their development of dementia. MCI is distinguished by memory impairment that is abnormal for the age and educational level of the individual. Although not all individuals with MCI develop AD, MCI can serve as a potential marker for early onset of AD. Some researchers have suggested that MCI be regarded as incipient AD and that individuals diagnosed with MCI would benefit from drug therapy (Sramek et al., Ann Pharmacother, 34(10):1179-88 (2000)). Thus, MCI screening may be beneficial in terms of early AD intervention and/or AD prevention.

### SUMMARY OF THE INVENTION

In one aspect, the invention provides a purified or isolated nucleic acid comprising a sequence that encodes a peptide loop corresponding to amino acid residues 136-216 of wild-type IRP-2 from humans, wherein said sequence comprises a mutation in said peptide loop, wherein said mutation interferes with the ability of a cysteine residue present in said peptide loop to undergo oxidation. In one embodiment, the nucleic acid sequence can comprise at least one of SEQ ID NOS: 3, 5, 7, 9, 11, 13, and 15. Preferably, the nucleic acid sequence encodes a peptide comprising a sequence selected from the group consisting of SEQ ID NOS: 4, 6, 8,10,12,14, and 16.

In another preferred embodiment, the purified or isolated polypeptide comprises a peptide loop corresponding to amino acid residues 136-216 of wild-type IRP-2 from humans, wherein said sequence comprises a mutation in said peptide loop, wherein said mutation interferes with the ability of a cysteine residue present in said peptide loop to undergo oxidation. The IRP-2 protein can comprise a sequence selected from the group consisting of SEQ ID NOS: 4, 6, 8, 10, 12, 14, and 16. Preferably, the IRP-2 protein is selected from the group consisting of SEQ. ID. Nos. SEQ ID NOS: 4, 6, 8, 10, 12, 14, and 16. More preferably, the invention concerns the use of such a mutant polypeptide in a method of making a probe for the diagnosis of a neurodegenerative disease and involves generating an antibody that binds to an epitope present on said mutant polypeptide, wherein said antibody does not cross react with a wild-type IRP-2 protein or fragment thereof. The mutant can comprise a substitution or a deletion of a cysteine residue. Further, the generating step can comprise culturing cells which produce said antibody.

In another embodiment, the invention concerns a method of identifying a subject in need of treatment or prevention of a neurodegenerative disease comprising: obtaining a biological sample from said subject having polynucleotides or protein; providing a probe, said probe being selected from the group consisting of a probe that interacts with a wild type or mutant IRP-2 protein and a probe that interacts with a polynucleotide encoding a wild type or mutant IRP-2 protein; contacting the biological sample with the probe under conditions that allow the probe to interact with the polynucleotide or protein in the biological sample;

detecting the amount of probe that interacts with the polynucleotide or protein in the biological sample; and identifying the subject as a subject in need of treatment or prevention of neurodegenerative disease by determining the presence or absence of the probe with the polynucleotide or protein in the biological sample. Preferably, the method comprises determining whether the probe interacts with the polynucleotide or protein in the biological sample. More preferably, the probe is selected from the group consisting of a nucleic acid, a protein, and a peptidomimetic. Further, the detection of the amount of probe that interacts with the polynucleotide or protein comprises use of a technique selected from the group consisting of fluorescence-activated cell sorting (FACs), immunoprecipitation, Western blot, immunochromatography, antibody staining, and a hybridization assay. Further, the neurodegenerative disease is Alzheimer's disease.

In another preferred embodiment, the invention concerns an antibody capable of specifically binding to a protein comprising an amino acid sequence selected from the group consisting of SEQ ID NOS: 4, 6, 8, 10, 12, 14, and 16. Preferably, the antibody specifically binds to a polypeptide comprising at least 10 consecutive amino acids of said protein and said protein has a mutation of a cysteine residue. More preferably, the antibody is a monoclonal antibody.

In another preferred embodiment, the invention concerns a purified or isolated antibody capable of specifically binding a mutant IRP-2 protein but does not specifically bind wild-type IRP-2 protein, wherein said mutant IRP-2 protein comprises a mutation in a peptide loop that corresponds to the amino acid sequence of SEQ ID NO: 2.

In another aspect, the invention concerns a method of differentiating mild cognitive impairment syndrome (MCI) from other forms of dementia in a human patient, comprising conducting magnetic resonance imaging (MRI) on the patient to quantitate and/or monitor brain iron wherein abnormal levels or distribution of brain iron indicate the presence of MCI.

### DETAILED DESCRIPTION OF THE INVENTION

One aspect of the invention relates to the discovery that mutations in the IRP-2 gene result in forms of IRP-2 proteins that resist degradation in the body and, thereby, perturb iron homeostasis. Some mutations occur within a peptide loop of IRP-2, wherein critical critical cysteine residues undergo an iron-dependent oxidation event that initiates the degradation process.

Embodiments include nucleic acids encoding mutant IRP-2 proteins, mutant IRP-2 proteins, and fragments of these molecules. Additionally, embodiments include nucleic acids that are complementary to nucleic acids encoding mutant IRP-2 proteins or fragments thereof and antibodies that bind mutant IRP proteins or fragments thereof. Preferably, the complementary nucleic acids described herein specifically detect a nucleic acid encoding a mutant IRP-2 protein and differentiate nucleic acids encoding a mutant IRP-2 protein from nucleic acids encoding a wild-type IRP-2 protein. Similarly, the preferred antibodies described herein specifically detect a mutant IRP-2 protein and differentiate a mutant IRP-2 protein from a wild-type IRP-2 protein.

Several assays described herein are designed to detect the presence of mutations in a nucleic acid encoding an IRP-2 protein or in the IRP-2 protein itself or a fragment of these molecules. Accordingly, nucleic acid sequences that complement nucleic acids that encode wild type and/or mutant IRP-2 proteins or fragments thereof and antibodies that bind epitopes on wild type and/or mutant IRP-2 proteins are used as ex vivo markers for neurodegenerative disease, including but not limited to, Alzheimer's disease. Thus, the diagnostic embodiments described herein concern both nucleic acid-based and protein-based assays and kits that incorporate these assays, which detect nucleic acids that encode a wild-type and/or mutant IRP-2 protein or IRP-2 proteins in biological samples (e.g., samples having peripheral blood cells). Automated techniques for diagnostic determination, such as standard flow cytometric techniques and array technology, can be used with some of the embodiments described herein. Monoclonal and polyclonal antibodies that detect wild-type or mutant IRP-2 proteins can be used with flow cytometry, for example, to rapidly determine whether a patient has a predilection to contract a neurodegenerative disease, such as Alzheimer's disease.

Support-based assays, such as ELISA, immunochromatography, and immunostrip assays, can also be adapted to detect the presence or absence of wild-type and/or mutant IRP-2 proteins. In one embodiment, for example, probes that bind to nucleic acids encoding wild-type or mutant IRP-2 proteins or antibodies that bind to mutant or wild-type IRP-2 proteins are joined to a support and are used to screen biological samples and, thereby, provide a diagnosis of a neurodegenerative disease.

Additionally, the diagnosis of neurodegenerative disease, such as Alzheimer's disease, can be accomplished by using wild type or mutant IRP-2 proteins or fragments thereof joined to a support. Accordingly, immobilized IRP-2 proteins or a fragment thereof is contacted with a biological sample having circulating antibodies and the presence or absence of antibodies to the mutant or wild-type IRP-2 protein can be determined by using a secondary detection molecule (e.g., a labeled anti-IgG antibody). The presence of antibody to mutant forms of IRP-2 indicates a predilection to contract a neurodegenerative disease.

It is contemplated that IRP-2 degradation and, thus the regulation of iron homeostasis, is initiated in healthy individuals by an iron-dependent oxidative modification that occurs at a peptide loop formed by amino acid residues 136-216 of IRP-2. This iron-dependent oxidation modifies three critical cysteine residues within this peptide loop and results in the production of aminomalonic acid. The conversion to aminomalonic acid sets the stage for ubiquitination, which signals proteosome degradation of IRP-2. In contrast, it is contemplated that individuals suffering from a neurodegenerative disease (e.g., Alzheimer's disease ) have mutations in the IRP-2 gene that result in IRP-2 proteins that are unable to undergo oxidative modification or exhibit a reduced level of oxidative modification. Some individuals may also have a multi-faceted gradient of IRP-2 proteins, wherein some IRP-2 proteins are unable to undergo iron-dependent oxidation, some IRP-2 proteins undergo a moderate amount of iron-dependent oxidation and other IRP-2 proteins undergo normal levels of iron dependent oxidation. By monitoring the levels of mutant and wild-type IRP-2 proteins and/or the nucleic acids encoding these molecules, a prognosis of neurodegenerative disease can be made.

Embodiments include nucleic acids encoding mutant IRP-2 proteins that are resistant to degradation in the body, complements thereto, and fragments of these proteins having at least one mutation. Desirably, these nucleic acids encode proteins that have mutations within a peptide loop corresponding to amino acid resides 136-216 of the sequence of human wild type IRP-2. A 189 nucleotide long fragment encoding a region of the wild type IRP-2 peptide loop is provided in the sequence listing. (SEQ ID NO: 1). The full -length cDNA sequence encoding human wild type IRP-2 is provided in SEQ ID NO: 17 and can be found in Guo et al., J. Biol. Chem. 270 16529 (1995), herein expressly incorporated by reference in its entirety. Additionally, the full-length cDNA sequence encoding rat wild type IRP-2 is provided in SEQ ID NO: 19 and can be found in Guo et al., J. Biol. Chem. 270 16529 (1995), herein expressly incorporated by reference in its entirety. When reference is made to wild type IRP-2 nucleic acids, depending of the context, it is meant to refer to the wild type IRP-2 molecules including those provided in SEQ. ID. Nos. 17 and/or 18 or that can be found in Guo et al., J. Biol. Chem. 270 16529 (1995), herein expressly incorporated by reference in its entirety.

Preferably, the nucleic acid embodiments have at least one mutation that results in an inability of a cysteine residue within the peptide loop corresponding to amino acid residues 136-216 of wild type human IRP-2 to undergo iron-dependent oxidation. This mutation may involve a substitution or deletion of a cysteine residue within this peptide loop or a mutation that perturbs the three-dimensional structure of the peptide loop so as to prevent iron-dependent oxidation. The sequences of several nucleic acids that encode a region of the peptide loop of a mutant IRP-2 protein are disclosed in SEQ ID NOS: 3, 5, 7, 9, 11, 13, and 15.

Some nucleic acid embodiments are genomic DNA, RNA, and cDNA encoding a mutant IRP-2, a complement thereto or a fragment of these molecules that contain at least one mutation. Some embodiments comprise a plurality of mutations that result in multiple substitutions and/or deletions within this peptide loop (e.g., mutations that result in the substitution and/or deletion of more than one cysteine). Preferably, the nucleic acid embodiments include the nucleotide sequences shown in the sequence listing (SEQ ID NOS: 3, 5, 7, 9, 11, 13, and 15 and complements thereof and/or fragments thereof. Nucleic acid sequences encoding mutant IRP-2 from humans, mammals, and other organisms are also embodiments, as are methods for obtaining such sequences. The nucleic acid embodiments can be altered, mutated, or changed such that the alteration, mutation, or change results in a conservative amino acid replacement.

The polypeptide embodiments described herein concern mutant forms of IRP-2 that are resistant to degradation in the body and fragments of these proteins having at least one mutation. Desirably such polypeptides have a mutation in a peptide loop corresponding to amino acid residues 136-216 of human wild type IRP-2, which contributes to the stability of the molecule to degradation in the body (e.g., stability to proteosome degradation.) A 63 amino acid long peptide corresponding to a region of the wild type IRP-2 peptide loop is provided in the sequence listing. (SEQ ID NO: 2). The full -length amino acid sequence of human wild type IRP-2 is provided in SEQ ID NO: 18 and can be found in Guo et al., J. Biol. Chem. 270 16529 (1995), herein expressly incorporated by reference in its entirety. Additionally, the full-length amino acid sequence of rat wild type IRP-2 is provided in SEQ ID NO: 20 and can be found in Guo et al., J. Biol. Chem. 270 16529 (1995), herein expressly incorporated by reference in its entirety. When reference is made to wild type IRP-2 proteins, depending of the context, it is meant to refer to the wild type IRP-2 proteins including those provided in SEQ. ID. Nos. 17 and/or 18 or that can be found in Guo et al., J. Biol. Chem. 270 16529 (1995), herein expressly incorporated by reference in its entirety.

Preferably, the polypeptide embodiments have at least one mutation that perturbs the iron-dependent oxidation of a cysteine residue within the peptide loop corresponding to amino acid residues 136-216 of human wild type IRP-2. This mutation may involve the substitution or deletion of a cysteine residue within this region or a mutation that perturbs the three-dimensional structure of the peptide loop so as to effect iron dependent oxidation of IRP-2. Some embodiments comprise a plurality of mutations within this peptide loop (e.g., more than one cysteine is mutated). Several mutant IRP-2 peptides are provided in SEQ ID NOS: 4, 6, 8, 10, 12, 14, and 16.

The polypeptide embodiments also include the partial or complete amino acid sequences shown in the sequence listing (SEQ ID NOS: 4, 6, 8, 10, 12, 14, and 16) and functional equivalents to such molecules including, but not limited to, the polypeptides of SEQ ID NOS: 4, 6, 8, 10, 12, 14, and 16 having non-conservative amino acid substitutions and peptidomimetics that resemble these molecules. Additional embodiments include methods of preparing the polypeptides described herein and molecules that bind these polypeptides. Embodiments also include, for example, polyclonal and monoclonal antibodies that recognize wild-type and/or mutant IRP-2. Preferred antibodies bind to epitopes on mutant IRP-2 but not wild-type IRP-2 or vice versa so as to distinguish between these molecules. Novel approaches to manufacture the monoclonal and polyclonal antibodies described herein are provided.

The diagnostic embodiments (including diagnostic kits) are designed to identify a predilection to neurodegenerative disease in organisms (e.g., insects, animals, mammals, and humans). Preferably, the diagnostic embodiments are employed to identify subjects at risk for Alzheimer's disease. Both nucleic acid and protein-based diagnostics are encompassed by aspects of this invention. That is, some diagnostic embodiments determine the predilection to neurodegenerative disease by detecting the presence or absence of a diagnostic nucleic acid or protein by using a probe that interacts said diagnostic nucleic acid or protein. The diagnostic nucleic acid can be, for example, a nucleic acid encoding a wild type or mutant IRP-2 protein or fragment thereof. The diagnostic protein can be, for example, a wild type or mutant IRP-2 protein or fragment thereof. The term “probe”, depending on the context, can refer to a molecule that interacts with a diagnostic nucleic acid or diagnostic protein or fragment thereof. Examples of “probes” include nucleic acids that complement at least a fragment of a wild type or mutant IRP-2 nucleic acid sequence (e.g., human or rat IRP-2) and antibodies that interact with epitopes that are present on a wild type or mutant IRP-2 protein sequence (e.g., human or rat IRP-2). Preferred probes specifically interact with said wild type diagnostic nucleic acid or diagnostic protein but not said mutant diagnostic nucleic acid or diagnostic protein or vice versa.

Some diagnostic embodiments, for example, concern support-bound assays that determine the ability of wild type or mutant IRP-2 or fragments thereof to interact with antibodies present in a biological sample. Desirably, the wild type or mutant IRP-2 or fragment thereof are disposed on the support in a multimeric fashion. Preferred embodiments comprise IRP-2 or a fragment thereof having a mutation in the peptide loop corresponding to amino acid residues 136-216 of wild type human IRP-2, which contributes to the stability of IRP-2. Most preferably, the IRP-2 or fragment thereof that is joined to the support to create the multimeric agent has at least one mutation that perturbs the ability of a cysteine residue within the peptide loop to undergo iron-dependent oxidation.

Embodiments also include diagnostic kits that can be used to identify a subject suffering from a neurodegenerative disease or a subject at risk of contracting a neurodegenerative disease. These diagnostic kits can include a nucleic acid that complements a nucleic acid that encodes a wild-type or mutant IRP-2 protein or an antibody that binds wild-type or mutant IRP-2 proteins (collectively referred to as “probes”). Additionally, the diagnostic kits can include various supports for immobilizing a sample, reagents, enzymes, detection chemicals, and instructions.

Some of the diagnostics approaches described herein identify defects in iron metabolism, which contribute to neurodegenerative phenotypes, such as AD. By detecting a polymorphism in a nucleic acid encoding an IRP-2 protein or in the IRP-2 protein itself, for example, a subject at risk of contracting a neurodegenerative disease can be identified. Other diagnostic approaches involve the detection of aberrant amounts or levels of a nucleic acid encoding a mutant IRP-2 protein or a mutant IRP-2 protein. By monitoring the levels of various polymorphic forms of IRP-2 protein a prognosis for neurodegenerative disease can be made. By one approach, the a ratio of wild-type IRP-2 to each mutant form of IRP-2 (or nucleic acids encoding these molecules) is made and, based upon a comparative analysis to the same ratios generated from healthy and diseased individuals, a prognosis for neurodegenerative disease is made. Additionally, ratios of wild type to total mutant form of IRP-2 can be generated and used to determine whether a subject is at risk of contracting a neurodegenerative disease. The section below describes several of the nucleic acid embodiments in greater detail.

Nucleic Acids Encoding Mutant IRP-2 Polypeptides

A family of mutant IRP-2 proteins have been discovered that can be identified by the presence of at least one mutation that perturbs the oxidation and concomitant degradation of the molecule. The nucleic acid embodiments of the invention include nucleotides encoding mutant IRP-2 proteins and fragments thereof. Some embodiments for example, include genomic DNA, RNA, and cDNA encoding these molecules. The nucleic acids encoding mutant IRP-2 proteins can be present in many different organisms including but not limited to insects, animals, and mammals.

The nucleotide sequences of the invention include, for example: (a) the DNA sequences shown in the sequence listing (SEQ ID NOS: 3, 5, 7, 9, 11, 13, and 15); (b) nucleotide sequences encoding the amino acid sequences shown in the sequence listing (SEQ ID NOS: 4, 6, 8, 10, 12, 14, and 16); (c) any nucleotide sequence that hybridizes to the complement of the DNA sequences shown in the sequence listing (EQ. ID Nos. 3, 5, 7, 9, 11, 13, and 15) under stringent conditions, e.g., hybridization to filter-bound DNA in 0.5 M NaHPO₄, 7.0% sodium dodecyl sulfate (SDS), 1 mM EDTA at 50° C. and washing in 0.2×SSC/0.2% SDS at 50° C.; and (d) any nucleotide sequence that hybridizes to the complement of the DNA sequences that encode an amino acid sequence provided in the sequence listing (SEQ ID NOS: 4, 6, 8, 10, 12, 14, and 16) under less stringent conditions (e.g., hybridization in 0.5 M NaHPO₄, 7.0% sodium dodecyl sulfate (SDS), 1 mM EDTA at 37° C. and washing in 0.2×SSC/0.2% SDS at 37° C.

Embodiments of the invention also include mutant IRP-2 nucleic acids that are isolated from other organisms (e.g., plants, molds, yeast, insects, animals, and mammals) whether naturally occurring or engineered. Approaches to isolate mutant IRP-2 nucleic acids in other species are provided infra. Embodiments also include fragments, modifications, derivatives, and variants of the sequences described above. Desired embodiments, for example, include nucleic acids having at least 9 consecutive bases unique to a mutant IRP-2 nucleic acid or a sequence complementary thereto and preferred fragments of the invention include at least 9 consecutive bases unique to a mutant IRP-2 nucleic acid or a sequence complementary thereto. In this regard, the nucleic acid embodiments can have from 9 to approximately 100 consecutive nucleotides. Some DNA fragments of the invention, for example, include nucleic acids having less than or equal to 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, and 240 consecutive nucleotides unique to a mutant IRP-2 nucleic acid and preferably encompass the region provided by the sequence of SEQ ID NOS: 3, 5, 7, 9, 11, 13, and 15 or a complement thereof. Preferably, the nucleic acid embodiments, however, comprise at least 12, 13, 14, 15, 16, 17, 18, or 19 consecutive nucleotides of a sequence unique to SEQ ID NOS: 3, 5, 7, 9, 11, 13, and 15 or complement thereof. More preferably, the nucleic acid embodiments comprise at least 20-30 consecutive nucleotides of a sequence unique to SEQ ID NOS: 3, 5, 7, 9, 11, 13, and 15 or complement thereof.

The nucleic acid embodiments can also be altered by mutation such as substitutions, additions, or deletions that provide for sequences encoding functionally equivalent molecules. Due to the degeneracy of nucleotide coding sequences, other DNA sequences that encode substantially the same mutant IRP-2 amino acid sequence as depicted in SEQ ID NOS: 4, 6, 8, 10, 12, 14, and 16 can be used in some embodiments. These include, but are not limited to, nucleic acid sequences comprising all or unique portions of a mutant IRP-2 nucleic acid or nucleic acids that complement all or unique parts of a mutant IRP-2 nucleic acid that has been altered by the substitution of different codons that encode a functionally equivalent amino acid residue within the sequence, thus producing a silent change, or a functionally non-equivalent amino acid residue within the sequence, thus producing a detectable change.

The nucleic acid sequences described above have biotechnological and diagnostic use, e.g., in nucleic acid hybridization assays, Southern and Northern Blot analysis, etc. and the prognosis of neurodegenerative disease (e.g., Alzheimer's disease). By using the nucleic acid sequences disclosed in the sequence listing SEQ ID NOS: 1, 3, 5, 7, 9, 11, 13, and 15, probes that complement wild type and/or mutant IRP-2 nucleic acids can be designed and manufactured by oligonucleotide synthesis. Desirable probes comprise a nucleic acid sequence that complements a nucleic acid sequence of SEQ ID NOS: 3, 5, 7, 9, 11, 13, and 15 that is unique to these molecules as compared to SEQ ID NO: 1. These probes can be used to screen cDNA or genomic libraries from various organisms (e.g., plants, molds, fungi, yeast, insects, animals, and mammals) so as to isolate natural sources of the nucleic acid embodiments. Screening can be by filter hybridization, for example, using duplicate filters. The labeled probe preferably contains at least 15-30 base pairs of a nucleic acid sequence that complements a nucleic acid sequence of (SEQ ID NOS: 3, 5, 7, 9, 11, 13, and 15) that is unique to these molecules as compared to SEQ ID NO: 1. The hybridization washing conditions used are preferably of a lower stringency when the cDNA library is derived from an organism different from the type of organism from which the labeled sequence is originated.

With respect to the cloning of a mutant IRP-2 nucleic acid, for example, hybridization can be performed in 0.5M NaHPO₄, 7.0% sodium dodecyl sulfate (SDS), 1 mM EDTA at 37° C. overnight and washing can be performed in 0.2×SSC/0.2% SDS at 37° C. Low stringency conditions are well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

Further, sequences from nucleic acids complementing a mutant or wild type IRP-2 nucleic acid or portions thereof, can be used to make oligonucleotide primers by conventional oligonucleotide synthesis for use in isolation and diagnostic procedures that employ the Polymerase Chain Reaction (PCR) or other enzyme-mediated nucleic acid amplification techniques. A mutant IRP-2 nucleic acid can be isolated from an organism of interest by performing PCR using two degenerate oligonucleotide primer pools designed on the basis of amino acid sequences within the mutant IRP-2 gene products disclosed herein. The template for the reaction can be cDNA obtained by reverse transcription of mRNA prepared from, for example, cells or tissue of an organism known or believed to express a mutant IRP-2 RNA. A variety of PCR techniques are familiar to those skilled in the art. For a review of PCR technology, see Molecular Cloning to Genetic Engineering White, B.A. Ed. in Methods in Molecular Biology 67: Humana Press, Totowa (1997), the disclosure of which is incorporated herein by reference in its entirety and the publication entitled “PCR Methods and Applications” (1991, Cold Spring Harbor Laboratory Press), the disclosure of which is incorporated herein by reference in its entirety.

For amplification of mRNAs, it is within the scope of the invention to reverse transcribe mRNA into cDNA followed by PCR (RT-PCR); or, to use a single enzyme for both steps as described in U.S. Pat. No. 5,322,770, the disclosure of which is incorporated herein by reference in its entirety. Another technique involves the use of Reverse Transcriptase Asymmetric Gap Ligase Chain Reaction (RT-AGLCR), as described by Marshall R. L. et al. (PCR Methods and Applications 4:80-84, 1994), the disclosure of which is incorporated herein by reference in its entirety. Briefly, RNA is isolated, following standard procedures, from an appropriate cellular or tissue source. A reverse transcription reaction is performed on the RNA using an oligonucleotide primer specific for the most 5′ end of the amplified fragment as a primer of first strand synthesis. The resulting RNA/DNA hybrid is then “tailed” with guanines using a standard terminal transferase reaction. The hybrid is then digested with RNAse H, and second strand synthesis is primed with a poly-C primer. Thus, cDNA sequences upstream of the amplified fragment are easily isolated. For a review of cloning strategies which can be used, see e.g., Sambrook et al., 1989, supra.

In each of these amplification procedures, primers on either side of the sequence to be amplified are added to a suitably prepared nucleic acid sample along with dNTPs and a thermostable polymerase, such as Taq polymerase, Pfu polymerase, or Vent polymerase. The nucleic acid in the sample is denatured and the primers are specifically hybridized to complementary nucleic acid sequences in the sample. The hybridized primers are then extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188, the disclosure of which is incorporated herein by reference in their entirety.

The primers are selected to be substantially complementary to a portion of the nucleic acid sequence of (SEQ ID NOS: 3, 5, 7, 9, 11, 13, and 15) that is unique to the mutant IRP-2 nucleic acid, thereby allowing the sequences between the primers to be amplified. Preferably, primers are 16, 17, 18, 19, 20, 21, 22, 23, 24, 25, 26, 27, 28, 29 and 30 nucleotides in length. The formation of stable hybrids depends on the melting temperature (Tm) of the DNA. The Tm depends on the length of the primer, the ionic strength of the solution and the G+C content. The higher the G+C content of the primer, the higher is the melting temperature because G:C pairs are held by three H bonds whereas A:T pairs have only two. The G+C content of the amplification primers of the present invention preferably ranges between 10 and 75%, more preferably between 35 and 60%, and most preferably between 40 and 55%. The appropriate length for primers under a particular set of assay conditions can be empirically determined by one of skill in the art.

The spacing of the primers relates to the length of the segment to be amplified. In the context of the present invention, amplified segments carrying nucleic acid sequence encoding fragments of a mutant IRP-2 nucleic acid can range in size from at least about 25 bp to 35 kb. Amplification fragments from 25-100 bp are typical, fragments from 50-200 bp are preferred and fragments from 200-300 bp are highly preferred. It will be appreciated that amplification primers can be of any sequence that allows for specific amplification of a region of a mutant IRP-2 nucleic acid and can, for example, include modifications such as restriction sites to facilitate cloning.

The PCR product can be subcloned and sequenced to ensure that the amplified sequences represent the sequences of a mutant IRP-2 gene. The PCR fragment can then be used to isolate a full length cDNA clone by a variety of methods. For example, the amplified fragment can be labeled and used to screen a cDNA library, such as a bacteriophage cDNA library. Alternatively, the labeled fragment can be used to isolate genomic clones via the screening of a genomic library. The identification and characterization of genomic clones from many different organisms (particularly humans) is helpful for designing diagnostic tests and clinical protocols for treating and preventing neurodegenerative disease.

Alternatively, a genomic library can be constructed using DNA obtained from an organism suspected of or known to carry the mutant IRP-2 allele, or a cDNA library can be constructed using RNA from a tissue known, or suspected, to express the mutant IRP-2 allele. The normal IRP-2 gene or any suitable fragment thereof can then be labeled and used as a probe to identify the corresponding mutant IRP-2 allele in such libraries. Preferably, however, the probes complement a sequence of SEQ ID NOS: 3, 5, 7, 9, 11, 13, and 15 that is unique to these mutant molecules. Clones containing the mutant IRP-2 gene sequences can then be purified and subjected to sequence analysis according to methods well known to those of skill in the art.

Additionally, an expression library can be constructed utilizing cDNA synthesized from, for example, RNA isolated from a tissue known, or suspected, to express a mutant IRP-2 allele in an organism suspected of, or known to carry, such a mutant allele. In this manner, gene products made by the putatively mutant cells can be expressed and screened using standard antibody screening techniques in conjunction with antibodies raised against the wild type or mutant IRP-2 gene product. (For screening techniques, see, for example, Harlow, E. and Lane, eds., 1988, Antibodies: A Laboratory Manual, Cold Spring Harbor Press, Cold Spring Harbor.) By using conventional antibody screening techniques, one can isolate wild type and/or mutant IRP-2 protein from expression libraries of various organisms. In cases where an IRP-2 mutation results in an expressed gene product with altered function (e.g., reduced oxidation of cysteine), a polyclonal set of antibodies against the mutant IRP-2 protein may react with the mutant gene product with high efficiency. Library clones detected via their reaction with such labeled antibodies can be purified and subjected to sequence analysis according to methods well known to those of skill in the art.

Embodiments also encompass (a) DNA vectors that contain any of the foregoing mutant IRP-2 coding sequences and/or their complements (i.e., antisense); (b) DNA expression vectors that contain any of the foregoing mutant IRP-2 coding sequences operatively associated with a regulatory element that directs the expression of the coding sequences; and (c) genetically engineered host cells that contain any of the foregoing mutant IRP-2 coding sequences operatively associated with a regulatory element that directs the expression of the coding sequences in the host cell. These recombinant constructs are capable of replicating autonomously in a host cell. Alternatively, the recombinant constructs can become integrated into the chromosomal DNA of a host cell. Such recombinant polynucleotides typically comprise a mutant IRP-2 genomic or cDNA polynucleotide of semi-synthetic or synthetic origin by virtue of human manipulation. Therefore, recombinant nucleic acids comprising mutant IRP-2 sequences and complements thereof that are not naturally occurring are provided herein.

Although nucleic acids encoding a mutant IRP-2 protein or nucleic acids having sequences that complement a mutant IRP-2 gene as they appear in nature can be employed, they will often be altered, e.g., by deletion, substitution, or insertion and can be accompanied by sequence not present in humans. As used herein, regulatory elements include, but are not limited to, inducible and non-inducible promoters, enhancers, operators and other elements known to those skilled in the art that drive and regulate expression. Such regulatory elements include, but are not limited to, the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

In addition, recombinant mutant IRP-2 nucleic acid sequences and their complementary sequences can be engineered so as to modify processing or expression of the protein. For example, and not by way of limitation, the mutant IRP-2 gene can be combined with a promoter sequence and/or ribosome binding site, or a signal sequence can be inserted upstream of coding sequence to permit secretion of the protein and thereby facilitate harvesting or bioavailability. Additionally, a given nucleic acid can be mutated in vitro or in vivo, to create and/or destroy translation, initiation, and/or termination sequences, or to create variations in coding regions and/or form new restriction sites or destroy preexisting ones, or to facilitate further in vitro modification. Any technique for mutagenesis known in the art can be used, including but not limited to, in vitro site-directed mutagenesis. (Hutchinson et al., J. Biol. Chem., 253:6551 (1978), herein incorporated by reference).

Further, nucleic acids encoding other proteins or domains of other proteins can be joined to nucleic acids encoding a mutant IRP-2 nucleic acid so as to create a fusion protein. Nucleotides encoding fusion protein embodiments can encode, for example, a full length mutant IRP-2 protein, a truncated mutant IRP-2 protein or a peptide fragment of an mutant IRP-2 protein fused to an unrelated protein or peptide, such as for example, glutathione; an Ig Fc domain, which increases the stability and half life of the resulting fusion protein; or an enzyme, fluorescent protein, luminescent protein which can be used as a marker (e.g., Green Fluorescent Protein (“GFP”)). The fusion proteins are also useful as biotechnological tools, as will be discussed infra. The section below describes several of the polypeptide embodiments and methods of making these molecules.

Mutant IRP-2 Polypeptides

Mutant IRP-2 polypeptides, fragments of these molecules, and chemicals that resemble these molecules including, but not limited to peptidomimetics, modified IRP-2 proteins, and derivatives or variants thereof are also embodiments. Mutant IRP-2 polypeptides can be present either naturally or through genetic engineering in a number of organisms (e.g., plants, insects, amphibians, reptiles, birds, other animals, cats, dogs, rodents, primates, humans, and other mammals).

The nucleic acids encoding a mutant IRP-2 protein or fragments thereof, described in the previous section, can be manipulated using conventional techniques in molecular biology so as to create recombinant constructs that express mutant IRP-2 protein or fragments of mutant IRP-2 protein. These polypeptides or derivatives thereof, include but are not limited to, those containing as a primary amino acid sequence all of the amino acid sequence substantially as depicted in the Sequence Listing (SEQ ID NOS: 4, 6, 8, 10, 12, 14, and 16) and fragments of SEQ ID NOS: 4, 6, 8, 10, 12, 14, and 16 at least three amino acids in length including altered sequences in which functionally equivalent amino acid residues are substituted for residues within the sequence resulting in a silent change. Preferred fragments of a sequence of SEQ ID NOS: 4, 6, 8, 10, 12, 14, and 16 are at least three amino acids and comprise amino acid sequence unique to mutant IRP-2 proteins including altered sequences in which functionally equivalent amino acid residues are substituted for residues within the sequence resulting in a silent change. The mutant IRP-2 peptide fragments can be, for example, less than or equal to 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 amino acids in length so long as said peptide has an amino acid that is unique to a mutant IRP-2 peptide, as compared to SEQ ID NO: 2.

Embodiments of the invention encompass proteins that are functionally equivalent to the mutant IRP-2 proteins encoded by the nucleotide sequences described in SEQ ID NOS: 4, 6, 8, 10, 12, 14, and 16, as judged by any of a number of criteria, including but not limited to the inability to be oxidized, the inability to be ubiquinated, and the ability to remain stable to proteosome degradation. Such functionally equivalent mutant IRP-2 proteins include, but are not limited to, additions or substitutions of amino acid residues within the amino acid sequence encoded by the mutant IRP-2 nucleotide sequences described above but, which result in a silent change, thus producing a functionally equivalent gene product. For example, embodiments include mutant IRP-2 proteins that have one or more amino acid residues within the mutant IRP-2 polypeptide of SEQ ID NOS: 4, 6, 8, 10, 12, 14, and 16 and fragments of SEQ ID NOS: 4, 6, 8, 10, 12, 14, and 16 that are substituted by another amino acid of a similar polarity that acts as a functional equivalent, resulting in a silent alteration. Substitutes for an amino acid within the sequence can be selected from other members of the class to which the amino acid belongs. For example, the non-polar (hydrophobic) amino acids include alanine, leucine, isoleucine, valine, proline, phenylalanine, tryptophan, and methionine. The polar neutral amino acids include glycine, serine, threonine, cysteine, tyrosine, asparagine and glutamine. The positively charged (basic) amino acids include arginine, lysine, and histidine. The negatively charged (acidic) amino acids include aspartic acid and glutamic acid. The aromatic amino acids include phenylalanine, tryptophan, and tyrosine.

The mutant IRP-2 polypeptides can be prepared by chemical synthesis methods (such as solid phase peptide synthesis) using techniques known in the art such as those set forth by Merrifield et al., J. Am. Chem. Soc. 85:2149 (1964), Houghten et al., Proc. Natl. Acad. Sci. USA, 82:51:32 (1985), Stewart and Young (Solid phase peptide synthesis, Pierce Chem Co., Rockford, Ill. (1984), and Creighton, 1983, Proteins: Structures and Molecular Principles, W. H. Freeman & Co., N.Y. herein incorporated by reference. Such polypeptides can be synthesized with or without a methionine on the amino terminus. Mutant IRP-2 proteins and fragments of thereof can be employed as biologically active or immunological substitutes for natural, purified mutant IRP-2 proteins and fragments of mutant IRP-2 proteins.

While the mutant IRP-2 proteins can be chemically synthesized, it can be more effective to produce these polypeptides by recombinant DNA technology using techniques well known in the art. Such methods can be used to construct expression vectors containing the mutant IRP-2 nucleotide sequences, for example, and appropriate transcriptional and translational control signals. These methods include, for example, in vitro recombinant DNA techniques, synthetic techniques, and in vivo genetic recombination. Alternatively, RNA capable of encoding an mutant IRP-2 nucleotide sequence can be chemically synthesized using, for example, synthesizers. See, for example, the techniques described in Oligonucleotide Synthesis, 1984, Gait, M. J. ed., IRL Press, Oxford, which is incorporated by reference herein in its entirety.

In several embodiments, mutant IRP-2 proteins and fragments of mutant IRP-2 proteins are expressed in a cell line. For example, some cells are made to express the IRP-2 polypeptide of SEQ ID NOS: 4, 6, 8, 10, 12, 14, and 16 or fragments of SEQ ID NOS: 4, 6, 8, 10, 12, 14, and 16. The sequences, constructs, vectors, clones, and other materials comprising these embodiments can advantageously be in enriched or isolated form. As used herein, “enriched” means that the concentration of the material is at least about 2, 5, 10, 100, or 1000 times its natural concentration (for example), advantageously 0.01%, by weight, preferably at least about 0.1% by weight. Enriched preparations from about 0.5%, 1%, 5%, 10%, and 20% by weight are also contemplated. The term “isolated” requires that the material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated. It is also advantageous that the sequences be in purified form. The term “purified” does not require absolute purity; rather, it is intended as a relative definition. Isolated proteins have been conventionally purified to electrophoretic homogeneity by Coomassie staining, for example. Purification of starting material or natural material to at least one order of magnitude, preferably two or three orders, and more preferably four or five orders of magnitude is expressly contemplated.

A variety of host-expression vector systems can be utilized to express the mutant IRP-2 proteins and fragments of mutant IRP-2 proteins. Where a mutant IRP-2 protein or fragment of mutant IRP-2 protein is a soluble derivative it can be recovered from the culture, i.e., from the host cell in cases where the peptide or polypeptide is not secreted, and from the culture media in cases where the peptide or polypeptide is secreted by the cells. However, the expression systems also encompass engineered host cells that express the mutant IRP-2 proteins and fragments of mutant IRP-2 proteins or functional equivalents in situ, i.e., anchored in the cell membrane. Purification or enrichment of the mutant IRP-2 protein or fragment thereof from such expression systems can be accomplished using appropriate detergents and lipid micelles and methods well known to those skilled in the art. However, such engineered host cells themselves can be used in situations where it is important not only to retain the structural and functional characteristics of the mutant IRP-2 protein, but to assess biological activity.

The expression systems that can be used include, but are not limited to, microorganisms such as bacteria (e.g., E. coli or B. subtilis) transformed with recombinant bacteriophage DNA, plasmid DNA or cosmid DNA expression vectors containing IRP-2 nucleotide sequences; yeast (e.g., Saccharomyces, Pichia) transformed with recombinant yeast expression vectors containing the mutant IRP-2 nucleotide sequences; insect cell systems infected with recombinant virus expression vectors (e.g., baculovirus) containing the mutant IRP-2 sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing mutant IRP-2 nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors can be advantageously selected depending upon the use intended for the mutant IRP-2 gene product being expressed. For example, when a large quantity of such a protein is to be produced, for the raising of antibodies to a wild type or mutant IRP-2 protein or fragment of wild type or mutant IRP-2 protein, for example, vectors that direct the expression of high levels of fusion protein products that are readily purified can be desirable. Such vectors include, but are not limited, to the E. coli expression vector pUR278 (Ruther et al., EMBO J., 2:1791 (1983), in which the mutant IRP-2 protein or fragment of mutant IRP-2 protein coding sequence can be ligated individually into the vector in frame with the lacZ coding region so that a fusion protein is produced; pIN vectors (Inouye & Inouye, Nucleic Acids Res., 13:3101-3109 (1985); Van Heeke & Schuster, J. Biol. Chem., 264:5503-5509 (1989)); and the like. pGEX vectors can also be used to express foreign polypeptides as fusion proteins with glutathione S-transferase (GST). In general, such fusion proteins are soluble and can be purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The mutant IRP-2 protein or fragment of mutant IRP-2 protein nucleic acid sequence can be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus, (i.e., virus lacking the proteinaceous coat coded for by the polyhedrin gene). These recombinant viruses are then used to infect Spodoptera frugiperda cells in which the inserted gene is expressed. (E.g., see Smith et al., J. Virol. 46: 584 (1983); and Smith, U.S. Pat. No. 4,215,051).

In mammalian host cells, a number of viral-based expression systems can be utilized. In cases where an adenovirus is used as an expression vector, the nucleotide sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the IRP-2 gene product in infected hosts. (E.g., See Logan & Shenk, Proc. Natl. Acad. Sci. USA 81:3655-3659 (1984)). Specific initiation signals can also be required for efficient translation of inserted mutant IRP-2 nucleotide sequences. These signals include the ATG initiation codon and adjacent sequences. In cases where an entire IRP-2 gene or cDNA, including its own initiation codon and adjacent sequences, is inserted into the appropriate expression vector, no additional translational control signals are needed.

However, in cases where only a portion of the mutant IRP-2 protein coding sequence is inserted, exogenous translational control signals, including, perhaps, the ATG initiation codon, should be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See Bittner et al., Methods in Enzymol., 153:516-544 (1987)).

In addition, a host cell strain can be chosen that modulates the expression of the inserted sequences, or modifies and processes the gene product in the specific fashion desired. Such modifications (e.g., glycosylation) and processing (e.g., cleavage) of protein products are important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, 3T3, and WI38.

For long-term, high-yield production of recombinant proteins, stable expression is preferred. For example, cell lines that stably express the wild type or mutant IRP-2 protein or fragment thereof can be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells are allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn are cloned and expanded into cell lines. This method is advantageously used to engineer cell lines that express the wild type or mutant IRP-2 proteins or fragments thereof.

A number of selection systems can be used, including but not limited to the herpes simplex virus thymidine kinase (Wigler, et al., Cell 11:223 (1977), hypoxanthine-guanine phosphoribosyltransferase (Szybalska & Szybalski, Proc. Natl. Acad. Sci. USA 48:2026 (1962), and adenine phosphoribosyltransferase (Lowy, et al., Cell 22:817 (1980) genes can be employed in tk⁻, hgprt⁻ or aprt⁻ cells, respectively. Also, antimetabolite resistance can be used as the basis of selection for the following genes: dhfr, which confers resistance to methotrexate (Wigler, et al., Proc. Natl. Acad. Sci. USA 77:3567 (1980); O'Hare, et al., Proc. Natl. Acad. Sci. USA 78:1527 (1981); gpt, which confers resistance to mycophenolic acid (Mulligan & Berg, Proc. Natl. Acad. Sci. USA 78:2072 (1981); neo, which confers resistance to the aminoglycoside G-418 (Colberre-Garapin, et al., J. Mol. Biol. 150:1 (1981); and hygro, which confers resistance to hygromycin (Santerre, et al., Gene 30:147 (1984)).

Alternatively, any fusion protein can be readily purified by utilizing an antibody specific for the fusion protein being expressed. For example, a system described by Janknecht et al. allows for the ready purification of non-denatured fusion proteins expressed in human cell lines. (Janknecht, et al., Proc. Natl. Acad. Sci. USA 88: 8972-8976 (1991)). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The mutant IRP-2 gene products or fragments thereof can also be expressed in plants, insects, and animals so as to create a transgenic organism. Plants and insects of almost any species can be made to express these molecules. Desirable transgenic plant systems having a wild type or mutant IRP-2 or fragment thereof include, for example, Arabadopsis, maize, and chlamydomonas. Desirable insect systems having a wild type or mutant IRP-2 or fragment thereof include, for example, D. melanogaster and C. elegans. Animals of any species, including, but not limited to, amphibians, reptiles, birds, mice, rats, rabbits, guinea pigs, pigs, micro-pigs, goats, dogs, cats, and non-human primates, e.g., baboons, monkeys, and chimpanzees can be used to generate a mutant IRP-2 transgenic animals. Transgenic organisms desirably exhibit germline transfer of mutant IRP-2 proteins or fragments thereof. Some transgenic organisms exhibit complete knockouts or point mutations of one or more existing IRP-2 genes. For example, in one embodiment, a transgenic animal comprises at least one point mutation at a cysteine residue within the peptide loop of IRP-2 corresponding to amino acid residues 136-216 and preferably within the region provided in SEQ. ID. NO: 2. The most preferred transgenic animal embodiments have mutations that resemble the mutant IRP-2 fragments provided in SEQ ID NOS: 4, 6, 8, 10, 12, 14, and 16.

Any technique known in the art is preferably used to introduce the mutant IRP-2 transgene into animals to produce the founder lines of transgenic animals or to knock out or replace existing IRP-2 genes. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al., Proc. Natl. Acad. Sci., USA 82:6148-6152 (1985); gene targeting in embryonic stem cells (Thompson et al., Cell 56:313-321 (1989); electroporation of embryos (Lo, Mol Cell. Biol. 3:1803-1814 (1983); and sperm-mediated gene transfer (Lavitrano et al., Cell 57:717-723 (1989); etc. For a review of such techniques, see Gordon, Transgenic Animals, Intl. Rev. Cytol. 115:171-229 (1989), which is incorporated by reference herein in its entirety.

The invention provides for transgenic animals that carry a mutant IRP-2 transgene in all their cells, as well as animals that carry the transgene in some, but not all their cells, i.e., mosaic animals. The transgene can be integrated as a single transgene or in concatamers, e.g., head-to-head tandems or head-to-tail tandems. The transgene can also be selectively introduced into and activated in a particular cell type by following, for example, the teaching of Lasko et al. (Lasko, M. et al., Proc. Natl. Acad. Sci. USA 89: 6232-6236 (1992)). The regulatory sequences required for such a cell-type specific activation will depend upon the particular cell type of interest, and will be apparent to those of skill in the art.

When it is desired that the mutant IRP-2 gene transgene be integrated into the chromosomal site of the endogenous mutant IRP-2 gene, gene targeting is preferred. Briefly, when such a technique is to be utilized, vectors containing some nucleotide sequences homologous to the endogenous mutant IRP-2 gene are designed for the purpose of integrating, via homologous recombination with chromosomal sequences, into and disrupting the function of the nucleotide sequence of the endogenous mutant IRP-2 gene. The transgene can also be selectively introduced into a particular cell type, thus inactivating the endogenous mutant IRP-2 gene in only that cell type, by following, for example, the teaching of Gu et al. (Gu, et al., Science 265: 103-106 (1994)). The regulatory sequences required for such a cell-type specific inactivation will depend upon the particular cell type of interest, and will be apparent to those of skill in the art.

Once transgenic animals have been generated, the expression of the recombinant mutant IRP-2 gene, for example, can be assayed utilizing standard techniques. Initial screening can be accomplished by Southern blot analysis or PCR techniques to analyze animal tissues to assay whether integration of the transgene has taken place. The level of mRNA expression of the transgene in the tissues of the transgenic animals can also be assessed using techniques which include, but are not limited to, Northern blot analysis of cells obtained from the animal, in situ hybridization analysis, and RT-PCR. Samples of mutant IRP-2 gene-expressing cells can also be evaluated immunocytochemically using antibodies specific for the mutant IRP-2 transgene product.

In addition to the naturally occurring polypeptide embodiments, derivative or modified molecules that produce a more desirable cellular response are within the scope of the invention. For example, a derivative mutant IRP-2 molecule can include a polypeptide that has been engineered to have one or more cysteine residues incorporated into the protein so as to promote the formation of a derivative that undergoes greater oxidation. The introduction of a cystine residue in a polypeptide can be accomplished using conventional molecular biology techniques.

Additional embodiments include peptidomimetics that resemble a mutant IRP-2 polypeptide. The naturally occurring amino acids employed in the biological production of peptides all have the L-configuration. Synthetic peptides can be prepared employing conventional synthetic methods, utilizing L-amino acids, D-amino acids, or various combinations of amino acids of the two different configurations. Synthetic compounds that mimic the conformation and desirable features of a particular peptide, e.g., an oligopeptide, once such peptide has been found, but that avoids the undesirable features, e.g., flexibility (loss of conformation) and bond breakdown are known as a “peptidomimetics”. (See, e.g., Spatola, A. F. Chemistry and Biochemistry of Amino Acids. Peptides, and Proteins (Weistein, B, Ed.), Vol. 7, pp. 267-357, Marcel Dekker, New York (1983), which describes the use of the methylenethio bioisostere [CH₂ S] as an amide replacement in enkephalin analogues; and Szelke et al., In peptides: Structure and Function, Proceedings of the Eighth American Peptide Symposium, (Hruby and Rich, Eds.); pp. 579-582, Pierce Chemical Co., Rockford, Ill. (1983), which describes renin inhibitors having both the methyleneamino [CH₂ NH] and hydroxyethylene [CHOHCH₂] bioisosteres at the Leu-Val amide bond in the 6-13 octapeptide derived from angiotensinogen).

In general, the design and synthesis of a peptidomimetic involves starting with the amino acid sequence of the peptide and conformational data (e.g., geometry data, such as bond lengths and angles) of a desired peptide (e.g., the most probable simulated peptide). That data is then used to determine the geometries that should be designed into the peptidomimetic. Numerous methods and techniques are known in the art for performing this step, any of which could be used. (See, e.g., Farmer, P. S., Drug Design, (Ariens, E. J. ed.), Vol. 10, pp. 119-143 (Academic Press, New York, London, Toronto, Sydney and San Francisco) (1980); Farmer, et al., in TIPS, 9/82, pp. 362-365; Verber et al., in TINS, 9/85, pp. 392-396; Kaltenbronn et al., in J. Med. Chem. 33: 838-845 (1990); and Spatola, A. F., in Chemistry and Biochemistry of Amino Acids. Peptides, and Proteins, Vol. 7, pp. 267-357, Chapter 5, “Peptide Backbone Modifications: A Structure-Activity Analysis of Peptides Containing Amide Bond Surrogates. Conformational Constraints, and Relations” (B. Weisten, ed.; Marcell Dekker: New York, pub.) (1983); Kemp, D. S., “Peptidomimetics and the Template Approach to Nucleation of □-sheets and α-helices in Peptides,” Tibech, Vol. 8, pp. 249-255 (1990). Additional teachings can be found in U.S. Pat. Nos. 5,288,707; 5,552,534; 5,811,515; 5,817,626; 5,817,879; 5,821,231; and 5,874,529. The section below describes the preparation and use of antibodies directed to wild type or mutant IRP-2 proteins or fragments thereof.

Anti-IRP-2 Antibodies

Following synthesis or expression and isolation or purification of an IRP-2 protein or a portion thereof, the isolated or purified protein can be used to generate monoclonal or polyclonal antibodies or both. Depending on the context, the term “antibodies” can encompass polyclonal, monoclonal, chimeric, single chain, Fab fragments and fragments produced by a Fab expression library. Antibodies that recognize a mutant or wild type IRP-2 protein or fragments thereof have many uses including, but not limited to, biotechnological applications, therapeutic/prophylactic applications, and diagnostic applications.

For the production of antibodies, various hosts including goats, rabbits, rats, mice, etc. can be immunized by injection with a mutant or wild type IRP-2 protein or any portion, fragment or oligopeptide that retains immunogenic properties. Depending on the host species, various adjuvants can be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin (KLH), and dinitrophenol. BCG (Bacillus Calmette-Guerin) and Corynebacterium parvum are also potentially useful adjuvants.

Peptides used to induce specific antibodies can have an amino acid sequence consisting of at least three amino acids, and preferably at least 10 to 15 amino acids. Preferably, short stretches of amino acids encoding fragments of a mutant or wild type IRP-2 protein are fused with those of another protein such as keyhole limpet hemocyanin (KLH) such that an antibody is produced against the chimeric molecule. While antibodies capable of specifically recognizing a mutant or wild type IRP-2 protein can be generated by injecting synthetic 3-mer, 10-mer, and 15-mer peptides that correspond to a protein sequence of a mutant or wild type IRP-2 protein into mice, a more diverse set of antibodies can be generated by using recombinant mutant or wild type IRP-2 protein or fragments thereof.

To generate antibodies to a mutant or wild type IRP-2 protein or fragments thereof, substantially pure protein is isolated from a transfected or transformed cell. The concentration of the polypeptide in the final preparation is adjusted, for example, by concentration on an Amicon filter device, to the level of a few micrograms/ml. Monoclonal or polyclonal antibody to the polypeptide of interest can then be prepared as follows:

Monoclonal antibodies can be prepared using any technique that provides for the production of antibody molecules by continuous cell lines in culture. These include, but are not limited to, the hybridoma technique originally described by Koehler and Milstein (Nature 256:495-497 (1975), the human B-cell hybridoma technique (Kosbor et al. Immunol Today 4:72 (1983); Cote et al Proc Natl Acad Sci 80:2026-2030 (1983), and the EBV-hybridoma technique Cole et al. Monoclonal Antibodies and Cancer Therapy, Alan R. Liss Inc, New York N.Y., pp 77-96 (1985). In addition, techniques developed for the production of “chimeric antibodies”, the splicing of mouse antibody genes to human antibody genes to obtain a molecule with appropriate antigen specificity and biological activity can be used. (Morrison et al. Proc Natl Acad Sci 81:6851-6855 (1984); Neuberger et al. Nature 312:604-608(1984); Takeda et al. Nature 314:452-454(1985). Alternatively, techniques described for the production of single chain antibodies (U.S. Pat. No. 4,946,778) can be adapted to produce single chain antibodies specific for a mutant or wild type IRP-2 protein. Antibodies can also be produced by inducing in vivo production in the lymphocyte population or by screening recombinant immunoglobulin libraries or panels of highly specific binding reagents as disclosed in Orlandi et al., Proc Natl Acad Sci 86: 3833-3837 (1989), and Winter G. and Milstein C; Nature 349:293-299 (1991).

Antibody fragments that contain specific binding sites for a mutant or wild type IRP-2 protein or fragments thereof can also be generated. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries can be constructed to allow rapid and easy identification of monoclonal Fab fragments with the desired specificity. (Huse W. D. et al. Science 256:1275-1281 (1989)).

By one approach, monoclonal antibodies are made as follows. Briefly, a mouse is repetitively inoculated with a few micrograms of the selected protein or peptides derived therefrom over a period of a few weeks. The mouse is then sacrificed, and the antibody producing cells of the spleen isolated. The spleen cells are fused in the presence of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol. 70:419 (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, New York. Section 21-2.

Polyclonal antiserum containing antibodies to heterogenous epitopes of a single protein can be prepared by immunizing suitable animals with the expressed protein or peptides derived therefrom described above, which can be unmodified or modified to enhance immunogenicity. Effective polyclonal antibody production is affected by many factors related both to the antigen and the host species. For example, small molecules tend to be less immunogenic than others and can require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J. Clin. Endocrinol. Metab. 33:988-991 (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980). Antibody preparations prepared according to either protocol are useful in quantitative immunoassays that determine concentrations of antigen-bearing substances in biological samples; they are also used semi-quantitatively or qualitatively (e.g., in diagnostic embodiments that identify the presence of a mutant or wild type IRP-2 protein in biological samples). An example of the preparation of antibodies specific for oxidized and reduced forms of wild type and mutant forms of IRP-2 is provided infra. The section below describes several IRP-2 characterization assays that evaluate the properties of wild type and mutant IRP-2 nucleic acids and proteins. Example 1 describes an approach that was used to make and screen antibodies that are specific for wild type and mutant IRP-2 and Example 2 describes a similar approach that was used to make and screen an antibody specific for wild type IRP-2.

IRP-2 Characterization Assays

The term “IRP-2 characterization assay” or “IRP-2 functional assay” or “functional assay” include assays that directly or indirectly evaluate the presence of a wild type or mutant IRP-2 nucleic acid or protein in a cell and the ability of wild type or mutant IRP-2 protein to associate with a membrane, interact with another molecule (e.g., ubiquitin), and/or undergo iron-dependent oxidation and proteosome degradation.

Some functional assays involve binding assays that utilize multimeric agents. One form of multimeric agent concerns a manufacture comprising a wild type or mutant IRP-2 protein or fragment thereof disposed on a support. These multimeric agents provide the wild type or mutant IRP-2 protein or fragment thereof in such a form or in such a way that a sufficient affinity is achieved. A multimeric agent having a n wild type or mutant IRP-2 protein or fragment thereof is obtained by joining the desired polypeptide to a macromolecular support. A “support” can be a termed a carrier, a protein, a resin, a cell membrane, or any macromolecular structure used to join or immobilize such molecules. Solid supports include, but are not limited to, the walls of wells of a reaction tray, test tubes, polystyrene beads, magnetic beads, nitrocellulose strips, membranes, microparticles such as latex particles, animal cells, DURACYTE®, artificial cells, and others. A wild type or mutant IRP-2 protein or fragment thereof can also be joined to inorganic carriers, such as silicon oxide material (e.g., silica gel, zeolite, diatomaceous earth or aminated glass) by, for example, a covalent linkage through a hydroxy, carboxy or amino group and a reactive group on the carrier.

In several multimeric agents, the macromolecular support has a hydrophobic surface that interacts with a portion of wild type or mutant IRP-2 protein or fragment thereof by a hydrophobic non-covalent interaction. In some cases, the hydrophobic surface of the support is a polymer such as plastic or any other polymer in which hydrophobic groups have been linked such as polystyrene, polyethylene or polyvinyl. Additionally, a wild type or mutant IRP-2 protein or fragment thereof can be covalently bound to carriers including proteins and oligo/polysaccarides (e.g. cellulose, starch, glycogen, chitosane or aminated sepharose). In these later multimeric agents, a reactive group on the molecule, such as a hydroxy or an amino group, is used to join to a reactive group on the carrier so as to create the covalent bond. Additional multimeric agents comprise a support that has other reactive groups that are chemically activated so as to attach the wild type or mutant IRP-2 protein or fragment thereof. For example, cyanogen bromide activated matrices, epoxy activated matrices, thio and thiopropyl gels, nitrophenyl chloroformate and N-hydroxy succinimide chlorformate linkages, or oxirane acrylic supports are used. (Sigma).

Furthermore, in some embodiments, a liposome or lipid bilayer (natural or synthetic) is contemplated as a support and wild type or mutant IRP-2 protein or fragment thereof are attached to the membrane surface or are incorporated into the membrane by techniques in liposome engineering. By one approach, liposome multimeric supports comprise a wild type or mutant IRP-2 protein or fragment thereof that is exposed on the surface. A hydrophobic domain can be joined to the wild type or mutant IRP-2 protein or fragment thereof so as to facilitate the interaction with the membrane.

The insertion of linkers, such as linkers (e.g., “λ linkers” engineered to resemble the flexible regions of λ phage) of an appropriate length between the wild type or mutant IRP-2 protein or fragment thereof and the support are also contemplated so as to encourage greater flexibility of the polypeptide of interest and thereby overcome any steric hindrance that can be presented by the support. The determination of an appropriate length of linker that allows for an optimal cellular response or lack thereof, can be determined by screening the wild type or mutant IRP-2 protein or fragment thereof with varying linkers in the assays detailed in the present disclosure.

In other embodiments, the multimeric supports discussed above can have attached multimerized wild type or mutant IRP-2 protein or fragments thereof so as to create a “multimerized-multimeric support”. A multimerized ligand can, for example, be obtained by coupling two or more polypeptides in tandem using conventional techniques in molecular biology. The multimerized form of the wild type or mutant IRP-2 protein or fragment thereof can be advantageous for many applications because of the ability to obtain an agent with a higher affinity, for example. The incorporation of linkers or spacers, such as flexible λ linkers, between the individual domains that make-up the multimerized agent can also be advantageous for some embodiments. The insertion of λ linkers of an appropriate length can encourage greater flexibility in the molecule and can overcome steric hindrance. Similarly, the insertion of linkers between the multimerized wild type or mutant IRP-2 protein or fragment thereof and the support can encourage greater flexibility and limit steric hindrance presented by the support. The determination of an appropriate length of linker can be determined by screening the wild type or mutant IRP-2 protein or fragment thereof with varying linkers with antibodies directed to epitopes on the wild type or mutant IRP-2 protein or fragment thereof. Example 3 describes an approach that was used to attach the anti-IRP antibodies, made according to Examples 1 or 2, to beads.

Thus, several approaches to identify agents that interact with a wild type or mutant IRP-2 protein or fragment thereof, employ the support-bound agents described above. Once the support-bound agent is obtained, for example, molecules (e.g., antibodies or ubiquitin) are contacted to the support-bound agent and an association is determined directly (e.g., by using labeled antibody or ubiquitin) or indirectly (e.g., by using a labeled antibody directed to the anti-IRP-2 antibody or ubiquitin). In some assays, it is desired to oxidize or reduce the support-bound wild type or mutant IRP-2 prior to contacting it with a binding partner such as ubiquitin. Such oxidation can be achieved in the presence of a sufficient concentration of iron (e.g., FeCl₃) although those of skill will appreciate many other ways of oxidizing a support-bound IRP-2 protein or fragment thereof. An approach to oxidize IRP-2 is provided in Iwai et al., Proc. Natl. Acad. Sci, USA, 95:4924 (1998), herein expressly incorporated by reference in its entirety.

In one characterization assay, for example, the ability of mutant support-bound IRP-2 peptides to undergo oxidation and ubiquitination is compared with the ability of wild type support-bound IRP-2 peptides to undergo oxidation and ubiquitination. By one method, oxidation of support bound IRP-2 is performed at the concentration of 0.1:g/:I protein in a 20:1 reaction mixture (25 mM Hepes-NaOH, pH 7.2 and 40 mM KCl) in the presence of 50 □M FeCl₃ and 10 mM DTT at 37° C. for 15-30 minutes. In some embodiments, it is desired to use Tris-carboxyethyl-phosphine (TCEP) at 1 mM to reduce the disulfides instead of DTT. In particular, when reduced IPR-2 is desired, preferably, TCEP at 1 mM is used in a reaction mixture without iron for 15-30 minutes at 37° C.

Once the oxidized and/or reduced IRP-2 supports are made, an in vitro ubiquitination assay can be performed as follows. The oxidized and/or reduced support-bound wild type and mutant IRP-2 is added to 400 □g RD4 S100 lysates, 5 mM MgCl₂, 2 mM ATP, 2 mM DTT, 6 □g ubiquitin, 25 mM Tris-Cl (pH 7.6) and 60 mM KCl for 5 minutes. Reactions are stopped by adding ice cold buffer containing 1% NP-40, 0.5% deoxycholate, 50 mM Tris-Cl (pH8.0), 150 mM NaCl, and 0.1% SDS. The support bound conjugate is washed in this buffer three times; the beads are spun down at 1500×g between washes. The beads are boiled for 10 minutes in 2× Laemmeli buffer and are separated on a suitable SDS PAGE (e.g., 6%-15%). The separated proteins are transferred to a membrane by electroblotting and the presence of ubiquitin can be verified by Western blotting with an affinity purified polyclonal or monoclonal anti-ubiquitin antobody. This assay will verify the ability of oxidized and reduced forms of mutant and wild type IRP-2 to interact with ubiquitin.

By another approach, the wild type and mutant support bound IRP-2 peptides are oxidized (e.g., exposure to H₂O₂ or iron) and the ability of the support bound agents to interact with radiolabeled ubiquitin is determined. Controls may include support bound agents that are reduced with TCEP. For example, aliquots of the support bound wild type and mutant IRP-2 proteins are exposed for 5, 10, 15, and 30 min to 0.02 mM, 0.05 mM, 0.07 mM, and 0.1 mM H₂0₂ in 50 mM Tris-HCl, pH 7.6, containing a mixture of inhibitors of proteinase and isopeptidase (5 mM EDTA, 10 μM hemin, 1 mM 4-(2-aminoethyl)benzene sulfonyl fluoride, 1 mM E-64, and 2 μg/ml aprotinin, and 10 mM iodoacetamide). Next, the assay is brought to a final volume of 50 μl, containing 50 mM Tris-HCl, pH 7.6, 5 mM MgCl₂, 1 mM DTT, 2 mM AMP-PNP, 2 μg of ¹²⁵I-ubiquitin at approx. 10⁶ cpm), 1 μM ubiquitin aldehyde, and 30 μl of support bound IRP-2 (approx. 10 mg of protein/ml).

Following incubation at 37° C. for 20 min, the support bound IRP2-ubiquitin conjugates are spun down at 1500×g for 30 seconds and washed in 50 mM Tris-HCl, pH 7.6, 5 mM MgCl₂, 1 mM DTT, 2 mM AMP-PNP. This washing procedure is repeated three times. The radioactivity associated with the support bound IRP-2 can be determined by scintillation. This approach directly detects the amount of ubiquitin that can associate with a mutant or wild type IRP-2 polypeptide.

Alternatively, the reaction above can be stopped by addition of 50 μl of 2× Laemmli buffer and boiling at 100° C. for 10 min. Subsequently, the proteins are separated on a 15% SDS-PAGE. The proteins are transferred to nylon by electroblot and the membrane is dried. The membrane is exposed to film for 2-4 days and, subsequently, a northern blot with an anti-IRP-2 antibody is performed. Detection of the bound antibody can be accomplished with a secondary antibody that is conjugated to gold or horse radish peroxidase, for example. In this manner, both ubiquitin and the IRP-2 proteins are detected. The level of ubiquitin conjugate can also be quantified by densitometry of the autoradiogram.

Additionally, a cell based characterization assay can be performed. For example, COS cells can be transfected to express mutant and/or wild type IRP-2 proteins. (See e.g., Samaniego et al., J. Biol. Chem. 269:30904 (1994), herein expressly incorporated by reference in its entirety, for a protocol for transfecting COS cells to express wild type IRP-2). After selecting transformants, aliquots of the positive expressing cells are placed under oxidative stress. By one approach, oxidative stress is brought about by raising the concentration of ferric ammonium citrate in the medium to 400 □g/ml. By another approach, exposure to oxidative stress is accomplished in a serum- and phenol red-free medium containing 0.1 mM H₂O₂ for 30 min. The cells are collected immediately or are cultured in H₂O₂ or iron-free medium to allow them to recover from oxidative stress. Control cells are treated exactly as the exposed cells except that H₂O₂ or iron are not included in the medium. The viability of the cells after exposure to H₂O₂ or iron can be monitored by exclusion of trypan blue and 3-(4,5-dimethyl thiazol-2-yl)-2,5-diphenyl tetrazolium bromide staining. The levels of reduced glutathione can also be determined. Further, the levels of ATP in the cells can be monitored using the bioluminescent somatic cell assay kit (Sigma) according to the manufacturer's instructions.

Cells are then harvested after the 30 min exposure to 0.1 mM H₂O₂ or iron and homogenized in 50 mM Tris-HCl, pH 7.6, containing a mixture of inhibitors of proteinase and isopeptidase (5 mM EDTA, 10 μM hemin, 1 mM 4-(2-aminoethyl)benzene sulfonyl fluoride, 1 mM E-64, and 2 μg/ml aprotinin, and 10 mM iodoacetamide). Following SDS-PAGE (8%) separation and transfer to nitrocellulose, the blots are probed with an affinity purified polyclonal antibody or monoclonal antibody to ubiquitin, followed by incubation with ¹²⁵I-protein A. The ubiquitin and ubiquitin conjugates are detected by autoradiography and quantified by image analysis.

Alternatively, the cells are harvested and homogenized in 50 mM Tris, 1 mM DTT, pH 7.6. Ubiquitin conjugation activity in the cell supernatant is quantified as the ability to catalyze the formation of conjugates between endogenous protein substrates and exogenous ¹²⁵I-labeled ubiquitin. This assay is done in a final volume of 50 μl, containing 50 mM Tris-HCl, pH 7.6, 5 mM MgCl₂, 1 mM DTT, 2 mM AMP-PNP, 2 μg of ¹²⁵I-ubiquitin approx. 10⁶ cpm), 1 μM ubiquitin aldehyde, and 30 μl of cell supernatant (10 mg of protein/ml). The reaction is started with addition of 30 μl of cell supernatant. Following incubation at 37° C. for20 min, the reaction is stopped by addition of 50 μl of 2× Laemmli buffer. After boiling at 100° C. for 10 min, proteins in 20 μl of the mixture are separated by a 15% SDS-PAGE. For a negative control, a parallel experiment is done in which AMP-PNP is replaced with 4.5 units of hexokinase and 12 mM 2-deoxyglucose. After drying the gel, it is exposed to film for 2-4 days. The level of ubiquitin conjugates can be quantified by densitometry of the autoradiogram. By employing the characterization assays described above, the ability of mutant and wildtype IRP-2 proteins to undergo oxidation and ubiquitination can be readily determined. (See also Shang et al., J. Biol. Chem. 272: 23086 (1997), herein expressly incorporated by reference in its entirety, for more ubiquitin assays that can be adapted for IRP-2-ubiquitin conjugate analysis.)

In light of the disclosure herein, one of skill will appreciate that such assays can be used to assess the ability of various forms of IRP-2 proteins to be selected for proteosome degradation and indicate forms of IRP-2 that are associated with neurodegenerative disease. In the disclosure below, several diagnostic embodiments are described.

### Diagnostic Embodiments

Generally, the diagnostic embodiments can be classified according to whether it is a nucleic acid or protein-based assay. Some diagnostic assays detect mutations or polymorphisms in IRP-2 nucleic acids or proteins, which contribute to aberrations in oxidation, ubiquitination, and proteosome degradation. Other diagnostic assays identify and distinguish defects in oxidation, ubiquitination, and proteosome degradation by detecting a level of mutant and/or wild type IRP-2 RNA or protein in a tested organism that resembles the level of mutant and/or wild type IRP-2 RNA or protein in a organism suffering from a disease or by detecting a level of mutant and/or wild type IRP-2 RNA or protein in a tested organism that is different than the level of mutant and/or wild type IRP-2 an organism not suffering from a disease.

Additionally, the manufacture of kits that incorporate the reagents and methods described in the following embodiments so as to allow for the rapid detection and identification of neurodegenerative disease are contemplated. The diagnostic kits can include a nucleic acid probe or an antibody or combinations thereof, which specifically detect a mutant or wild type form of IRP-2 nucleic acid or protein or a nucleic acid probe or an antibody or combinations thereof, which can be used to determine the level of RNA or protein expression of a wild type or mutant IRP-2. The detection component of these kits will typically be supplied in combination with one or more of the following reagents. A support capable of absorbing or otherwise binding DNA, RNA, or protein will often be supplied. Available supports include membranes of nitrocellulose, nylon or derivatized nylon that can be characterized by bearing an array of positively charged substituents. One or more restriction enzymes, control reagents, buffers, amplification enzymes, non-human polynucleotides like calf-thymus or salmon-sperm DNA, and a set of instructions that describe how to diagnose a neurodegeneratve disease (e.g., Alzheimer's disease) with the tools in the kit can also be supplied.

Useful nucleic acid-based diagnostic techniques include, but are not limited to, direct DNA sequencing, Southern Blot analysis, single-stranded confirmation analysis (SSCA), RNAse protection assay, dot blot analysis, nucleic acid amplification, and combinations of these approaches. The starting point for these analysis is isolated or purified nucleic acid from a biological sample. It is contemplated that blood from a subject would be a suitable biological sample. Further, if the diagnostic assay is designed to determine the presence of a mutant or polymorphic IRP-2, any source of DNA including, but not limited to hair, cheek cells and skin cells can be used as a biological sample. The nucleic acid is extracted from the sample and can be amplified by a DNA amplification technique such as the Polymerase Chain Reaction (PCR) using primers that correspond to regions flanking DNA that encodes amino acid residues recognized as a polymorphism that contributes to a defect in oxidation, ubiquitination, and proteosome degradation, thus, providing a prognosis of neurodegenerative disease.

Once a sufficient amount of DNA is obtained from an individual to be tested, several methods can be used to detect an IRP-2 polymorphism. Direct DNA sequencing, either manual sequencing or automated fluorescent sequencing can detect such sequence variations. Another approach is the single-stranded confirmation polymorphism assay (SSCA) (Orita et al., Proc. Natl. Acad. Sci. USA 86:2776-2770 (1989), herein expressly incorporated by reference in its entirety). This method, however, does not detect all sequence changes, especially if the DNA fragment size is greater than 200 base pairs, but can be optimized to detect most DNA sequence variation.

The reduced detection sensitivity is a disadvantage, but the increased throughput possible with SSCA makes it an attractive, viable alternative to direct sequencing for mutation detection. The fragments that have shifted mobility on SSCA gels are then sequenced to determine the exact nature of the DNA sequence variation. Other approaches based on the detection of mismatches between the two complementary DNA strands include clamped denaturing gel electrophoresis (CDGE) (Sheffield et al., Am. J. Hum. Genet. 49:699-706 (1991)), heteroduplex analysis (HA) (White et al., Genomics 12:301-306 (1992)), and chemical mismatch cleavage (CMC) (Grompe et al., Proc. Natl. Acad. Sci. USA 86:5855-5892 (1989)). A review of currently available methods of detecting DNA sequence variation can be found in Grompe, Nature Genetics 5:111-117 (1993).

Seven well-known nucleic acid-based methods for confirming the presence of a polymorphism are described below. Provided for exemplary purposes only and not intended to limit any aspect of the invention, these methods include:
(1) single-stranded confirmation analysis (SSCA) (Orita et al.);
(2) denaturing gradient gel electrophoresis (DGGE) (Wartell et al., Nucl. Acids Res. 18:2699-2705 (1990) and Sheffield et al., Proc. Natl. Acad. Sci. USA 86:232-236 (1989)), both references herein incorporated by reference;
(3) RNAse protection assays (Finkelstein et al., Genomics 7:167-172 (1990) and Kinszler et al., Science 251:1366-1370 (1991)) both references herein incorporated by reference;
(4) the use of proteins which recognize nucleotide mismatches, such as the E. Coli mutS protein (Modrich, Ann. Rev. Genet. 25:229-253 (1991), herein incorporated by reference;
(5) allele-specific PCR (Rano and Kidd, Nucl. Acids Res. 17:8392 (1989), herein incorporated by reference), which involves the use of primers that hybridize at their 3′ ends to a polymorphism and, if the polymorphism is not present, an amplification product is not observed; and
(6) Amplification Refractory Mutation System (ARMS), as disclosed in European Patent Application Publication No. 0332435 and in Newton et al., Nucl. Acids Res. 17:2503-2516 (1989), both references herein incorporated by reference; and
(7) temporal temperature gradient gel electrophoresis (TTGE), as described by Bio-Rad in U.S./E.G. Bulletin 2103, herein incorporated by reference.

In SSCA, DGGE, TTGE, and RNAse protection assay, a new electrophoretic band appears when the polymorphism is present. SSCA and TTGE detect a band that migrates differentially because the sequence change causes a difference in single-strand, intramolecular base pairing, which is detectable electrophoretically. RNAse protection involves cleavage of the mutant polynucleotide into two or more smaller fragments. DGGE detects differences in migration rates of sequences using a denaturing gradient gel. In an allele-specific oligonucleotide assay (ASOs) (Conner et al., Proc. Natl. Acad. Sci. USA 80:278-282 (1983)), an oligonucleotide is designed that detects a specific sequence, and an assay is performed by detecting the presence or absence of a hybridization signal. In the mutS assay, the protein binds only to sequences that contain a nucleotide mismatch in a heteroduplex between polymorphic and non-polymorphic sequences. Mismatches, in this sense of the word refers to hybridized nucleic acid duplexes in which the two strands are not 100% complementary. The lack of total homology results from the presence of one or more polymorphisms in an amplicon obtained from a biological sample, for example, that has been hybridized to a non-polymorphic strand. Mismatched detection can be used to detect point mutations in DNA or in an mRNA. While these techniques are less sensitive than sequencing, they are easily performed on a large number of biological samples and are amenable to array technology.

In some embodiments, nucleic acid probes that differentiate polynucleotides encoding wild type IRP-2 from mutant IRP-2 are attached to a support in an ordered array, wherein the nucleic acid probes are attached to distinct regions of the support that do not overlap with each other. Preferably, such an ordered array is designed to be “addressable” where the distinct locations of the probe are recorded and can be accessed as part of an assay procedure. These probes are joined to a support in different known locations. The knowledge of the precise location of each nucleic acid probe makes these “addressable” arrays particularly useful in binding assays. The nucleic acids from a preparation of several biological samples are then labeled by conventional approaches (e.g., radioactivity or fluorescence) and the labeled samples are applied to the array under conditions that permit hybridization.

If a nucleic acid in the samples hybridizes to a probe on the array, then a signal will be detected at a position on the support that corresponds to the location of the hybrid. Since the identity of each labeled sample is known and the region of the support on which the labeled sample was applied is known, an identification of the presence of the polymorphic variant can be rapidly determined. These approaches are easily automated using technology known to those of skill in the art of high throughput diagnostic or detection analysis.

Additionally, an opposite approach to that presented above can be employed. Nucleic acids present in biological samples can be disposed on a support so as to create an addressable array. Preferably, the samples are disposed on the support at known positions that do not overlap. The presence of nucleic acids having a desired polymorphism in each sample is determined by applying labeled nucleic acid probes that complement nucleic acids that encode the polymorphism and detecting the presence of a signal at locations on the array that correspond to the positions at which the biological samples were disposed. Because the identity of the biological sample and its position on the array is known, the identification of the polymorphic variant can be rapidly determined. These approaches are also easily automated using technology known to those of skill in the art of high throughput diagnostic analysis.

Any addressable array technology known in the art can be employed with this aspect of the invention. One particular embodiment of polynucleotide arrays is known as GENECHIPS™, and has been generally described in U.S. Pat. No. 5,143,854; PCT publications WO 90/15070 and 92/10092. These arrays are generally produced using mechanical synthesis methods or light directed synthesis methods, which incorporate a combination of photolithographic methods and solid phase oligonucleotide synthesis. (Fodor et al., Science, 251:767-777, (1991)). The immobilization of arrays of oligonucleotides on solid supports has been rendered possible by the development of a technology generally identified as “Very Large Scale Immobilized Polymer Synthesis” (VLSPIS™) in which, typically, probes are immobilized in a high density array on a solid surface of a chip. Examples of VLSPIS™ technologies are provided in U.S. Pat. Nos. 5,143,854 and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and diagnostic information. Examples of such presentation strategies are disclosed in PCT Publications WO 94/12305, WO 94/11530, WO 97/29212, and WO 97/31256.

A wide variety of labels and conjugation techniques are known by those skilled in the art and can be used in various nucleic acid assays. There are several ways to produce labeled nucleic acids for hybridization or PCR including, but not limited to, oligolabeling, nick translation, end-labeling, or PCR amplification using a labeled nucleotide. Alternatively, a nucleic acid encoding an IRP-2 can be cloned into a vector for the production of an mRNA probe. Such vectors are known in the art, are commercially available, and can be used to synthesize RNA probes in vitro by addition of an appropriate RNA polymerase such as T7, T3 or SP6 and labeled nucleotides. A number of companies such as Pharmacia Biotech (Piscataway N.J.), Promega (Madison Wis.), and U.S. Biochemical Corp (Cleveland Ohio) supply commercial kits and protocols for these procedures. Suitable reporter molecules or labels include those radionuclides, enzymes, fluorescent, chemiluminescent, or chromogenic agents, as well as, substrates, cofactors, inhibitors, magnetic particles and the like.

The RNAse protection method, briefly described above, is an example of a mismatch cleavage technique that is amenable to array technology. Preferably, the method involves the use of a labeled riboprobe that is complementary to an IRP-2 sequence having a polymorphism. However, the method can involve the use of a labeled riboprobe that is complementary to an IRP-2 sequence having the wild type gene. The riboprobe and either mRNA or DNA isolated and amplified from a biological sample are annealed (hybridized) and subsequently digested with the enzyme RNAse A, which is able to detect mismatches in a duplex RNAse structure. If a mismatch is detected by RNAse A, the polymorphic variant is not present in the sample and the enzyme cleaves at the site of the mismatch and destroys the riboprobe. Thus, when the annealed RNA is separated on a electrophoretic gel matrix, if a mismatch has been detected and cleaved by RNAse A, an RNA product will be seen which is much smaller than the full length duplex RNA for the riboprobe and the mRNA or DNA.

Complements to the riboprobe can also be dispersed on an array and stringently probed with the products from the Rnase A digestion after denaturing any remaining hybrids. In this case, if a mismatch is detected and probe destroyed by Rnase A, the complements on the array will not anneal with the degraded RNA under stringent conditions. In a similar fashion, DNA probes can be used to detect mismatches, through enzymatic or chemical cleavage. See, e.g., Cofton, et al., Proc. Nat. Acad. Sci. USA 85:4397 (1988); Shenk et al., Proc. Natl. Acad. Sci. USA 72:989 (1975); and Novack et al., Proc. Natl. Acad. Sci. USA 83:586 (1986). Mismatches can also be detected by shifts in the electrophoretic ability of mismatched duplexes relative to matched duplexes. (See, e.g., Cariello, Human Genetics 42:726 (1988), herein incorporated by reference). With any of the techniques described above, the mRNA or DNA from a tested organism that corresponds to regions of an IRP-2 having a polymorphism can be amplified by PCR before hybridization.

The presence of an IRP-2 polymorphism or wild type sequence in a protein sample can also be detected by using conventional assays. For example, antibodies immunoreactive with an IRP-2 polymorphism can be used to screen biological samples for the predilection of a neurodegenerative disease (e.g., Alzheimer's disease). Additionally, antibodies that differentiate the wild type IRP-2 from mutant IRP-2 can be used to determine that an organism does not have a predilection of a neurodegenerative disease (e.g., Alzheimer's disease). In preferred embodiments, antibodies are used to immunoprecipitate the wildtype or mutant forms of IRP-2 from solution or are used to react with the wild type or mutant IRP-2 on Western or Immunoblots. Favored diagnostic embodiments also include enzyme-linked immunosorbant assays (ELISA), radioimmunoassays (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Other embodiments employ aspects of the immune-strip technology disclosed in U.S. Pat. Nos. 5,290,678; 5,604,105; 5,710,008; 5,744,358; and 5,747,274, herein expressly incorporated by reference in their entireties.

In another preferred protein-based diagnostic, antibodies of the invention are attached to a support in an ordered array wherein a plurality of antibodies are attached to distinct regions of the support that do not overlap with each other. As with the nucleic acid-based arrays, the protein-based arrays are ordered arrays that are designed to be “addressable” such that the distinct locations are recorded and can be accessed as part of an assay procedure. These probes are joined to a support in different known locations. The knowledge of the precise location of each probe makes these “addressable” arrays particularly useful in binding assays. For example, an addressable array can comprise a support having several regions to which are joined a plurality of antibody probes that specifically recognize a particular IRP-2 protein and differentiate the mutant and wild type IRP-2 proteins.

Accordingly, proteins are obtained from biological samples and are labeled by conventional approaches (e.g., radioactivity, colorimetrically, or fluorescently). The labeled samples are then applied to the array under conditions that permit binding. If a protein in the sample binds to an antibody probe on the array, then a signal will be detected at a position on the support that corresponds to the location of the antibody-protein complex. Since the identity of each labeled sample is known and the region of the support on which the labeled sample was applied is known, an identification of the presence, concentration, and/or expression level can be rapidly determined. That is, by employing labeled standards of a known concentration of mutant or wild-type IRP-2 protein, an investigator can accurately determine the protein concentration of the particular IRP-2 protein in a tested sample and can also assess the expression level of the specific form of IRP-2 protein. Conventional methods in densitometry can also be used to more accurately determine the concentration or expression level of the specific IRP-2 protein. These approaches are easily automated using technology known to those of skill in the art of high throughput diagnostic analysis.

In another embodiment, an opposite approach to that presented above can be employed. Proteins present in biological samples can be disposed on a support so as to create an addressable array. Preferably, the protein samples are disposed on the support at known positions that do not overlap. The presence of a protein encoding a mutant or wild-type IRP-2 protein in each sample is then determined by applying labeled antibody probes that recognize epitopes specific for the mutant or wild-type form of IRP-2 protein. Because the identity of the biological sample and its position on the array is known, an identification of the presence, concentration, and/or expression level of a particular polymorphism can be rapidly determined.

That is, by employing labeled standards of a known concentration of mutant and/or wild-type IRP-2 protein, an investigator can accurately determine the concentration of IRP-2 protein in a sample and from this information can assess the expression level of the particular form of IRP-2 protein. Conventional methods in densitometry can also be used to more accurately determine the concentration or expression level of the IRP-2 protein. These approaches are also easily automated using technology known to those of skill in the art of high throughput diagnostic analysis. As detailed above, any addressable array technology known in the art can be employed with this aspect of the invention and display the protein arrays on the chips in an attempt to maximize antibody binding patterns and diagnostic information.

In another diagnostic embodiment, the immune-strip technology disclosed in U.S. Pat. Nos. 5,290,678; 5,604,105; 5,710,008; 5,744,358; and 5,747,274, herein expressly incorporated by reference in their entireties, is adapted to present an antigen that is recognized by antibodies to wild type or mutant IRP-2 protein. These antigen presenting immunostrips are then used to analyze biological samples for the presence of antibodies to the various forms of IRP-2 protein. Although wild type or mutant IRP-2 peptides or protein are the preferred antigen for these embodiments, peptidomimetics that resemble these molecules can be used. These peptidomimetic-based embodiments can be more protease resistant and may be stripped and used for many applications. Preferably, peptidomimetic-based IRP-2 arrays are created (e.g., genechips having a peptidomimetic resembling wild type and mutant IRP-2 protein) and used to screen large panels of biological samples.

In another preferred approach, blood samples from subjects suspected as being at risk for a neurodegenerative disease are obtained and analyzed by flow cytometry (FACS) using antibodies directed to epitopes on the wild type IRP-2 protein and/or mutant forms of IRP-2 protein. Standard flow cytometric techniques using fluorescently labeled secondary antibodies (e.g., fluorescin conjugated goat anti-human IgG) and commercially available cell fixation and permeabilization kits (PermaCyte-FP) will be used. Accordingly, resuspended cells are reacted with the anti-IRP-2 antibody and the secondary antibody and the immune complexes are passed before the FACS. Cells will be monitored for the distribution and quantitation of fluorescence. By using antibodies specific for wildtype and/or mutant IRP-2 proteins, a determination of the presence and amount of various forms of IRP-2 proteins can be rapidly determined.

As discussed above, the presence or detection of a polymorphism in an IRP-2 molecule can provide a diagnosis of a neurodegenerative disease (e.g., Alzheimer's disease). Additional embodiments include the preparation of diagnostic kits comprising detection components, such as antibodies, specific for a particular polymorphic variant of IRP-2. The detection component will typically be supplied in combination with one or more of the following reagents. A support capable of absorbing or otherwise binding RNA or protein will often be supplied. Available supports for this purpose include, but are not limited to, membranes of nitrocellulose, nylon or derivatized nylon that can be characterized by bearing an array of positively charged substituents, and Genechips™ or their equivalents. One or more enzymes, such as Reverse Transcriptase and/or Taq polymerase, can be furnished in the kit, as can dNTPs, buffers, or non-human polynucleotides like calf-thymus or salmon-sperm DNA. Results from the kit assays can be interpreted by a healthcare provider or a diagnostic laboratory. Alternatively, diagnostic kits are manufactured and sold to private individuals for self-diagnosis.

In addition to diagnosing disease according to the presence or absence of a polymorphism in an IRP-2 DNA, mRNA, or protein, some neurodegenerative diseases involving defects in oxidation, ubiquitination, and proteosome degradation of IRP-2 result from skewed levels of mutant and wildtype IRP-2. By monitoring the level of expression of specific forms of IRP-2, for example, a diagnosis can be made or a disease state can be identified. That is, many neurodegenerative diseases result from a dosage effect, wherein an overabundance of a mutant IRP-2 that is unable to undergo oxidation persists. Thus, by determining ratios of the level of expression of various IRP-2 (e.g., patterns of IRP-2 expression) a prognosis of health or disease can be made.

Accordingly, the levels of IRP-2 expression in various samples from healthy individuals, as well as, individuals suffering from a neurodegenerative disease is determined. These values can be recorded in a database and can be compared to values obtained from tested individuals. Additionally, the ratios or patterns of IRP-2 expression in various samples from both healthy and diseased individuals are recorded in a database. These analyses are referred to as “disease state profiles” and by comparing one disease state profile (e.g. from a healthy or diseased individual) to a disease state profile from a tested individual, a clinician can rapidly diagnose the presence or absence of disease. Databases having measurements of IRP-2 expression of several individuals afflicted with a neurodegenerative disease are valuable standards by which the progression of disease can be monitored. In this manner, deviation between the standard and the organism values establishes the severity of disease state.

The nucleic acid and protein-based diagnostic techniques described above can be used to detect the level or amount or ratio of expression of a IRP-2 RNAs or proteins in a tissue. Through quantitative Northern hybridizations, In situ analysis, immunohistochemistry, ELISA, genechip array technology, PCR, and Western blots, for example, the amount or level of expression of RNA or protein for a particular IRP-2 (wild-type or mutant) can be rapidly determined and from this information ratios of expression can be ascertained. One diagnostic approach, for example, involves a method of correlating the ratio between the expression levels of a plurality of IRP-2 isoforms with a disease state. To practice this method, biological samples from individuals suffering from a neurodegenerative disease and biological samples from normal individuals are obtained. Next, the expression levels of two or more IRP-2 proteins or nucleic acids encoding IRP-2 proteins (e.g., a wild type and a mutant form of IRP-2) in the samples is determined and an analysis as to whether there is a statistically significant association between the ratio of wild type and mutant IRP-2 expression and the neurodegenerative disease is made. Statistically significant associations can be determined using statistical methods familiar to those skilled in the art, including t test and chi-square analyses.

Once the levels of various IRP-2 molecules are determined, the information can be recorded onto a computer readable media, such as a hard drive, floppy disk, DVD drive, zip drive, etc. After recording and the generation of a database comprising the levels of expression of the various IRP-2 molecules studied, a comparing program is used which compares the levels of expression of the various IRP-2 molecules so as to create a ratio of expression. In a first comparison, for example, a wild type IRP-2 to a mutant IRP-2 ratio is generated. Additionally, desirable comparisons can include, but are not limited to, the various mutant forms of IRP-2 with each other and/or wild type IRP-2. The examples describe the preparation of antibodies specific for oxidized and reduced wild type and mutant forms of IRP-2.Another aspect of the invention relates to a method for using magnetic resonance imaging (MRI) to measure and/or monitor brain iron in its various in vivo states (non-transferrin bound iron (NTBI), transferrin bound iron (TBI), and high molecular weight complexes, including ferritin and hemosiderin). The integration of high resolution 3D gradient echo imaging, phase imaging (SWI) with removal of background field effects, understanding T2* signal losses, and the extraction of susceptibility using a special spin echo/gradient echo imaging method leads to absolute quantification of brain iron.

A. High Resolution 3D, Gradient Echo Imaging

Obtaining a sensitivity to small local field susceptibility effects requires imaging with long echo times. Background field effects caused by air/tissue interfaces lead to dramatic signal losses in tissues adjacent to these areas for long echo times. Dephasing across a voxel, as previously shown, can be reduced by using very small voxels so that phase variation from background (or other) fields is reduced to less than 2π. This maneuver leads to a dramatic recovery of signal and, when the images are filtered, to recover the lost signal-to-noise going to higher resolution. These results are different than what would have been obtained with lower resolution. This method is referred to as the commutator effect, i.e., a special non-linearity inherent in the MR acquisition methods. Phase images from echo times as long as 120 ms at 1.5 T have been shown to represent a means to visualize susceptibility differences between tissues. Venous structures, gray matter/white matter susceptibility differences and iron in the basal ganglia become visible with this modification.

B. Phase Imaging and Removing Background Field Effects

The use of phase images is a natural way to begin evaluating the presence of paramagnetic (or diamagnetic) differences between tissues in the brain. The phase in an MR image is given by (p=γΔBt

where γ is the gyromagnetic ratio, ΔB the change in magnetic field from one tissue to the next, and t the time at which the data are measured (usually the echo time TE).

The problem with visualizing these differences arises from the extra phase effects from background field effects from air/tissue interfaces. A high pass phase filter was developed to remove the background field effects that are predominantly low spatial frequency. Phase images dramatically improved with this processing technique. This method has been successfully used to map oxygen saturation in the brain and even small changes in oxygen saturation during functional brain activation (that is, we could easily measure changes as small as 0.03 ppm with an error of 0.002 for the particular 3D sequence used for those experiments). This implies that, for a p value of 0.025, it is asserted that susceptibility differences as small as 0.004 are measurable with this sequence (The sequence used was a 5 minute, 3D gradient echo method with a TE=40 ms and a resolution of 0.5×1.0×2.0 mm³). Sensitivity can be improved by collecting the data a second time and averaging it, or averaging multiple acquisitions as well as filtering the data to a lower resolution.

Another approach to remove all linear phase effects is to use a double echo method. The phase from the first echo is used to predict the phase from the second echo based upon the simple linear dependence of phase expected from background phase effects. When the phase of the first echo, γΔBTE1, is multiplied by TE2/TE1 to predict γΔBTE2, and the predicted phase subtracted from that of the second echo (accomplished by complex division), the expected phase of the corrected image is zero and leaves behind any non-linear effects associated with a two-compartment model where the phase effects are not simply additive. This approach makes it possible to separate small local pixel effects.

C. Understanding T2* Signal Losses

Recently, analytical evaluation of the signal loss in a spherical voxel caused by a dipole field the source of which was a small volume enclosed within the voxel has been performed. Long echo times can induce significant oscillations in the signal behavior (i.e., signal changes that are non-exponential in nature). In an attempt to quantify signal losses in T2* weighted imaging, a theory was developed that allows prediction of signal behavior in the presence of small randomly distributed local sources of susceptibility change. Specifically, signal change is not exponential in nature near the time origin and is in the intermediate (and long) time domains. The theory can be used to extract the volume susceptibility of the random sources as well as their volume fraction within a voxel. The exact quantitative nature of this method has not been validated for a set of random structures in vivo.

The dependence of T1 relaxation in the brain was studied and revealed that frontal areas in the brain have the longest T1 for gray matter while areas near the motor cortex have the smallest T1 values. A loss of contrast with gray matter and white matter in this region results. Phase measurements of the same regions reveal a strong correlation between phase and iron content, showing the largest in the motor cortex. The present invention involves correlation between the phase and iron content in the brain as a function of age.

D. Extraction of Susceptibility Using a Special Spin Echo/Gradient Echo Imaging Method

In order to account for background field inhomogeneities across a slice or voxel not caused by microscopic effects, and to eliminate any dependence on the rf pulse design as well, a special combined gradient echo and spin echo acquisition method was defined. A series of gradient echoes are collected around the spin echo and used to remove background field effects as follows. The last gradient echo is divided by the first gradient echo image to create the equivalent of a T2 weighted image without dephasing across the voxel. Once T2 is known, the effect of T2 on the multiple gradient echoes is removed so that only the pristine changes due to T2′ (those signal changes caused by the local sources of susceptibility) remain. The signal dependence can be shown to have a quadratic behavior in time and, when fit, both the magnetic moment and volume content of the sources can be extracted quantitatively.

In a preferred embodiment of the invention, MR imaging will be done at 1.5 T and 4.7 T to study humans and animals. Susceptibility differences as small as 0.004 ppm can be measured with a 3D gradient echo method with a TE=40 ms and a resolution of 0.5×1.0×2.0 mm³ (requiring a 5 minute scan time). By going to 4.7 T and using a small mouse brain surface coil, a factor of roughly 8 in SNR is gained and reduced the voxel size to 0.5 mm×0.5 mm×0.5 mm. If data is collected for 40 minutes, the voxel size can be reduced to 0.25 mm×0.25 mm×0.25 mm which is not critical to the success of this project, but provides benefits in differentiating structures in the mouse brain.

Another preferred embodiment involves magnetic image (MRI) analysis of material referred to as phantoms with a known susceptibility to validate the ability of MRI to correctly quantify iron content with a given geometry. Three different shapes are considered: first, a simple test tube is imaged both parallel and perpendicular to the main field; second, a thin plane is imaged; and third a thin plane warped to represent more the folds in the brain parenchyma is imaged. These planes are created using mylar film to separate layers 2 to 3 mm thick to mimic the human brain. The dimensions of these phantoms are on the order of 10 cm on each side again to mimic the human brain and also to allow proper tuning and shimming of the phantoms. An agarose gel is used as the filling material doped with several different iron carrying compounds with a series of different concentrations (first 100 nmol/gm and then 4 more times starting at 500 nmol/gm at increments of 500 nmol/gm up to 2000 nmol/gm) to mimic the iron in the brain. The susceptibility of each compound is measured using the test tube shape since the effect of the geometry on phase under these conditions is well understood. Specifically, four different types of phantoms composed of a) FeCl—, b) FeSO4, c) ferritin, and d) hemosiderin are prepared. The phantom experiments allow study of the effect of concentration of iron for random systems (low concentrations are expected to have an exponential effect on signal loss while high concentrations are expected to be more geometry dependent). However, the structure and iron concentration of the brain do not appear to have the usual geometry effect as the sulci phase and is found to be rather uniform, independent of the folding that occurs. This may indicate a low concentration and, hence, an easy means to calibrate the phase independent of object shape. These experiments are carried out at both 1.5 T and 4.7 T to ensure that the linear behavior is present and that there are no special calibrations required from one field strength to the next. Accordingly, imaging of phantoms allows correlation of the susceptibility measurements from the MR images to the known iron concentration within each of the phantoms. Phantoms of different shapes and with different concentrations of various iron molecules can be tested. Using the phantoms, sequence optimization is performed at both 1.5 T for humans and 4.7 T for animals prior to implementation. The present invention involves a method of iron quantification for use on animals and humans and for monitoring iron changes over time in the Alzheimer's brain.

Another preferred embodiment involves magnetic resonance image analysis of transgenic mice that accumulate brain iron and sustain a neurodegenerative disease for validation of the MRI technique. Transgenic mice engineered with a deletion encoding the iron regulatory protein-2 (IRP2) are known to accumulate significant levels of neuronal iron (La Vaute 2001). The transgenic mice have onset of neurodegenerative symptoms within 6 months of age that is typically manifest as ataxia, vestibular dysfunction tremors and postural abnormalities among others (La Vaute 2001). Preferably, mice are kept on a twelve-hour light-dark schedule with free access to commercial pellet chow and water. Further, mice are observed for skin, oral mucosal, behavioral and neurologic signs, and weighed weekly. Animals showing abnormal behaviors are to be euthanized. At selected intervals, animals are anesthetized for neuro-imaging by isoflurane inhalation (4% induction, 1% maintenance) and then placed into an MRI compatible stereotactic apparatus. Rectal temperature is monitored continuously and maintained at 37±0.5° C. with a warm water coil placed under the animal.

In order to precisely correlate the novel imaging findings using the optimized MRI sequences, four groups of mice are imaged. The present invention involves the monitoring of iron regulated proteins, including IRP-2, both by quantity and localization in mouse brain tissue. The groups will be a) controls (C57bI/6), b) Ireb2+/+, c) Ireb2+/, and d) Ireb2−/− mice. The groups will provide a comprehensive overview of the effect of the accumulation of iron (and its various forms) within the brain. Previous work has demonstrated that these mice progressively increase brain iron content up to 18 months of age. Using the optimized sequences developed, mice are imaged consecutively at 1, 3, 6, 9, 12 and 18 months to understand the spatial and temporal deposition of iron within the brain. The initial imaging group is sufficiently large to allow extraction of 6 animals at each time point for quantitative histochemistry which allows absolute verification of the various forms of iron within the brain (and other tissues), correlates the imaging data with the actual levels of iron and correlates the regional levels of iron and the imaging data which provides definitive insights into the basis for degenerative diseases.

Prior to imaging, mice are anesthetized with isoflurane to a sufficient level to prevent movement artifacts on MR images. Imaging is performed on a Bruker Avance 4.7 T imager with a head only volume coil. After homogenizing the magnetic field, a spin echo T1-weighted scout is obtained with a relaxation time (TR) of 700 ms and an echo time (TE) of 20 ms with 2 acquisitions and 3 slices in the coronal, sagittal and transverse directions. Ten slices, each 2 mm thick with a 2 mm separation is positioned at the level of the hippocampus (˜4 mm posterior to bregma) and piriform cortex (at the level of bregma) on the scout images. Spin echo diffusion weighted and multi-echo T2-weighted data sets are collected. The parameters used for the diffusion-weighted sequences are 2200/100 ms (TRITE) for coronal slices with 2 acquisitions with a 45 mm field of view (FOV) and a 128×128 matrix. The diffusion gradients are applied in the z direction. A b value of 1000 s/mm² is used to compute the apparent diffusion coefficient (ADC) maps. Multi-echo T2-weighted sequence parameters consisted of 3000/40 ms (TR/TE) and 6 echos each 40 ms apart, with one acquisition for 3-5 slices.

The ADC map is determined by the equation: ADC=In(So/Sn)/b where Sn is the mean intensity for a DW image and So is the mean intensity for the corresponding diffusion unweighted image {6032}. ADCs are calculated for each pixel in the map. High ADC values are represented as bright on DW maps. ADCs for regions of interest (ROI) are calculated as the mean of the ADC for all pixels in the specified area. T2 maps are generated from 6 echo T2 sequences. T2 relaxation constants are then calculated for each pixel using nonlinear least squares curve fit to the data using the equation: M(t)=Mo(1−e^{-t/T2}) where Mo is the initial magnetization value before decay, t is the echo time (ms) and T2 is the spin-spin relaxation time.

Another preferred embodiment concerns image analysis performed for each mice on a single slice immediately anterior to the slice where the hippocampus can be seen curling inferiorly. This position corresponded approximately to bregma −3.60 mm and maximized the cross-sectional area of each region of interest (ROI). CHESHIRE™ image processing software (Hayden Image Processing Group, Waltham, Mass.) is used to outline and analyze the ROI's that are confirmed by a second researcher. The bilateral ROI's included the amygdala (and associated nuclei), piriform cortex (including part of the entorhinal and perirhinal cortices), hippocampus, retrosplenial cortex (including motor and somatosensory cortices) and thalamus. A two pixel width separates the hippocampi and retrosplenial ROI's. A line is drawn across the bottom of both hippocampi that extends across the cortex demarcated the inferior border of the retrosplenial ROI. The piriform and amygdala ROI's are abutted each other and extended the same distance superiorly and inferiorly. Medially two to four pixels separate the thalamus from the amygdaloid ROI, to minimize signal contribution from the lateral ventricle. A 5 by 5 pixel square is centered within the thalamus. For the 3D gradient echo images, information from slices around this slice is evaluated.

Although the ability to extract iron from phantom data is promising, iron quantitation in the animal (and human) brain are confounded by innate ambiguities. For a chemical differentiation of iron types, the relative susceptibilities as seen by MRI between tissues and iron in ferritin, macrophages and free iron around beta-amyloid plaques are attempted. The brain iron are quantified using phase and T2′ imaging and compared to that found histo-chemically in the mouse brain. It is well known that there is iron in and around the blood vessels near the plaque. The MR images are correlated with the brain iron changes in the mouse model, and provide the potential to detect early plaque formation.

In another embodiment of the invention, upon completion of MRI analysis, four mice are euthanized for brain and blood tissue harvest, storage and processing for use in immuno-histochemistry and iron chemistry that complement the MRI studies and in combination allow understanding of the role of IRP-2 in brain iron metabolism. The blood and brain tissue are removed from the mice after CO₂ asphyxiation. The blood is pooled per group and processed to isolate leukocytes and to obtain a serum sample archived frozen at −70° C. The brains are separated into right and left hemispheres, weights recorded by wet weight, and randomly, one hemi-brain from each animal is placed in pip (4% buffered formalin solution) and the other half placed in cryoprotectant and frozen. The frozen brain tissue is sectioned and every third section from the front (level 1), middle (level 2), and the back (level 3) is placed on poly-l-lysine coated slides. Cerebral cortex, lateral ventricles, corpus callosum and caudate putamen are present in level 1 sections, cerebral cortex, thalamus, third ventricle and hippocampus are present in level 2 sections, and cerebellum, medulla, fourth ventricle and pyramidal tracts are present in level 3 sections. Thirty-three 5 to 10 micron sections are made from each half-brain (in triplicate: 1 set for H&E, 1 set for apoptosis, 1 set for prussian blue staining for ferric iron distribution, and 7 sets for immunocytochemistry using anti-IRP-1, anti-IRP-2, anti-ferritin, anti-transferrin receptor, β-amyloid ubiquitin and hemosiderin). Three additional 30 μm sections (1 from each brain level) are weighed (wet weight) and placed into evacuated freezer bags for processing elemental iron content.

For H&E and apoptosis measurements of the mice tissues, one 5-10 μm-tissue section from each of the three brain levels is stained with H&E for morphologic assessment of the tissue. One set of each brain tissue sections is labeled using an Apodirect assay modified as described by Green et al. 2001. DNA damage is used to assess late apoptotic events in the brain tissue using terminal deoxynucleotidyl-transferase (TdT) mediated fluorescent (FITC)-conjugated BrdU incorporation into free 3′ ends of nucleic acids. Briefly, tissue is fixed in −20° C. 70% ethanol for 15 minutes. The fixed tissue is re-hydrated in PBS for five minutes and incubated with a mixture of TdT, reaction buffer and FITC-BrdU provided with the kit. Tissue is incubated with the DNA labeling mixture overnight at room temperature (22-24° C.), washed and counter-stained for 30 minutes with propidium iodide (PI)/RNAse, washed and protected with permafluor and covered with glass coverslips. FITC-BrdU incorporation is quantified using a laser scanning cytometer (LSC) (CompuCyte, Cambridge, Mass.) as described below.

For immunocytochemical labeling of frozen tissue sections of the mice, tissue is fixed as described above for the apoptosis assay (Green et al. 1995; Green et al. 2001). The fixed tissue is labeled with primary antibodies (anti-Irp-1, anti-Irp-2, anti-ferritin, anti-transferrin/transferrin receptor, □-amyloid ubiquitin and hemosiderin). Incubation with primary antibodies/antisera is 16 hrs at 4° C., followed by washing in PBS containing 0.05% Tween-20 (PBST). Antibodies that are not directly conjugated to a fluorescent molecule are secondarily labeled with alexa-488, alexa-594, Cy-2 or Cy-5 anti-mouse or rabbit IgG antibodies for a minimum incubation period of 4 hrs at 25° C. Just prior to completion of the secondary antibody incubation, DAPI (1 μg/ml) or propidium iodide (PI, 5 μg/ml), depending on the ability to dual label and/or confocal microscopy as the analytical endpoint, is added for 10 min. Excess secondary antibody and nuclear counterstain is removed by washing in PBST, and the tissue is protected with permafluor and glass coverslips and is dried flat in the dark prior to quantitative analysis (LSC and Confocal microscopy as described below) and photography. Non-specific fluorescence is determined by incubating control sections with non-immune sera and secondary antibody, or secondary antibody alone.

For quantification of specific proteins, including IRP-2, in the mice tissues, Laser Scanning Cytometric (LSC) is performed on fluorescently-labeled mice tissues. The LSC has an Olympus BX50 base and is configured with argon ion, helium-neon and UV lasers for 6-color analysis. There are four sensors, simultaneously digitized at 625,000 Hz corresponding nominally to 0.5-micrometer spatial intervals along the scan. Fluorescent energy is collected by the objective, reflected by a partially silvered mirror to allow the CCD camera to image the cells and steered through a scan lens to the scanning mirror. Dichroic mirrors and optical interference filters are supported by 4 photo-multiplier tubes, each capable of detecting a specific range of fluorescent wavelengths. The fluorescent measurements and x, y coordinates are recorded digitally and stored as FCS files in the computer base. Tissue to be analyzed is contoured by the labeled nucleus. A variety of gating parameters can be chosen, but includes those that collect information on signal intensity versus cell size, cell number (area, perimeter, count, etc). Protocol settings and display parameters are optimized using control positive and negative samples, the optimized protocol and display files are stored and utilized in scanning replicate sections.

For localization of the specific proteins quantitated by the LSC, confocal microscopy is performed on the mice tissues. Many proteins have discrete locations that coincide with their functional properties, and thus, a better understanding is gained by the cellular/subcellular localization of specific proteins by confocal microscopy (Altura et al. 2001). Fluorescently labeled tissue sections are imaged in 3-dimension using an Olympus IX-70 based, BioRad-1024 confocal microscope. Sections are acquired with low power (4-20×) in 0.5 μm z-steps for general distribution, and high power (40-100×) magnification for acquiring cellular/subcellular location of the proteins listed above.

For iron quantification of mice tissues, iron distribution is estimated by, Prussian blue staining (ferric) and total iron by atomic spectroscopy. Prussian blue staining is performed on paraffin embedded sections using a sensitive modification of the standard method (Moos & Mollgard, 1993) which involves mixing potassium ferrocyanide (2.5%) with HCL acid (2.5%) for 20 minutes at room temperature, rinsed with hydrogen peroxide and cell nuclei counterstained with PI.

For statistical analysis of the MR imaging data, left and right comparison of the bilateral region of interests (ROI) will be performed for each animal using a one way ANOVA (p<0.05). A two-tailed student's t-test (significant at p<0.05, highly significant at p<0.01) will then be performed to compare the control values with the experimental values at each time point.

To extract relative iron content from the MR imaging data, the present invention involves an imaging method that uses a high resolution 3D gradient echo sequence. Most susceptibility features to be studied are best imaged when the resolution is a factor of one to two times the size of the object. For this reason, in the human brain, the following parameters: TR=67 ms, TE=40 ms, resolution of 0.5×1.0×2.0 mm³ with a scan time of 5 minutes were used. Small structures like venules on the order of 300 to 500 microns and small iron deposits less than 1 mm³ in size in the basal ganglia are visible in the image because of their signal cancellation properties and their phase effects in the image. This exquisite sensitivity to micro-voxel effects makes SWI so powerful. As mentioned above, for a 40 minute scan, a resolution of 0.25×0.25×0.25 mm³ can be collected in the mouse brain.

In another embodiment of the invention, for a random system, voxel size is less important and then larger voxels can be used for faster imaging and better SNR. The gradient echo sequence is tested on the phantoms mentioned above to measure the phase as a function of iron concentration and as a function of resolution to ensure scale invariance of the measurement.

Since current iron concentration differences between gray matter and white matter are manifest as roughly 20 degrees in 40 ms which corresponds to roughly 0.1 ppm, error analysis of phase in phantoms, animals and humans was applied. If the SNR in the magnitude image is only 15:1 then the standard deviation of the phase is 0.06 radians (40 or 0.002 ppm) or roughly a ten percent error. One could then imagine evaluating susceptibility differences greater than 3 standard deviations which corresponds to 0.006 ppm with a p value of 0.005. Improvement of data by averaging can be accomplished in two ways. First, more data is acquired or the data is filtered, depending on the resolution required. For example, if a lower resolution is sufficient, then the SNR can be dramatically improved per unit time by running the experiment with lower resolution to enhance the SNR. For example, an MR scan with the resolution of 0.5×1.0×2.0 mm³ and a TE=40 ms takes 5 minutes to acquire and yields the values quoted above. However, if a resolution of 1×2×2 mm³ is sufficient, then the scan takes half the time and yields 2 times higher SNR. So if the same 5 minutes were used, the increase in SNR is 2sqrt(2) tantamount to imaging at a field strength 8 times higher. Resolutions may vary from 0.25 microns to 1 mm in the phantoms and animals.

For human studies, several sets of imaging parameters on the first 5 normals and 5 age matched Alzheimer's patients are used. Echo times of 40, 80 and 120 ms in order to evaluate the in vivo sensitivities are included. The resolution may vary from 0.5×0.5×1.0 mm³ to 1×1×2 mm³ in order to study the sensitivity as a function of voxel size. On the higher resolution images, the data will be filtered to a lower resolution image by a factor of two in each direction to compare with the lower resolution data. Reasons for this method include the fact that Gibbs ringing is reduced relative to the lower resolution scan, the effects of field inhomogenities are reduced, and scale invariance effects can be checked.

An absolute iron content can be extracted from the MR imaging data. A linear increase in T2′ measurements is predicted as iron concentration increases. A multi-echo, gradient/spin echo combination defined as follows is used. A spin echo structure with a TE of 80 ms is created. About this echo time, a series of 31 echoes of the same polarity (an echo every 2.5 ms) is collected. The following theory (see ref xx, xy and xz for more details) has been theoretically predicted and experimentally verified. The signal behavior for a random set of spheres (which is an excellent approximation for iron known to conglomerate in spherical shapes) especially given the large voxels we are using) is given by:
S(t)=ρ(1−λ)exp(−0.4|(tδω)²) for tδω<1.5
and
S(t)=ρ(1−λ)exp(−itΔω)exp(−R2′abs(t−tₛ)) for tδω>1.5
where
δω=γ4π(M−M₀)/3, R2′=1.21 λδω, tₛ=1/(1.21 δω) and Δω=−0.16 λδω.

By measuring the short time and long time components, a numerical estimate for the arguments in each exponential and, therefore, for (M−M₀) (extracted from dw) and for λ (extracted from R2′ since δω is now known) is obtained. For example, for magnetic particulates used as contrast agents in MRI, such as AMI 225, δω has the value 3.4×10⁻⁷/s. Using a volume fraction of A=2×10⁶ yields an R2′ of 82.23/s in excellent agreement with other estimates (which range from 80 to 100/s from numerical simulations). When the susceptibility is known, R2′ can be used directly as a measure of the volume fraction λ. For much smaller susceptibilities on the order of ppm, such as diamagnetic or paramagnetic substances, δω is on the order of ms. For example, for a vein with haematocrit of 0.4 and oxygen saturation of 55%, the value of δω is about 3 ms. Ironically, the smaller the susceptibility, the longer δω and when tₛ lies between 3 and 30 ms after or before the spin echo, the signal can now be used to find the susceptibility of the source producing the signal loss as well as its volume content. Because the susceptibilities of each of the components found to be responsible for the signal loss (ferritin, etc.) are measured, this feature is not used. However, another numerical estimate can be made for the phase term −0.16 λδωwhich is directly related to R2′. This estimate adds no new information when there is only one source of magnetic field variation. However, if non-heme iron is not the only source and heme iron contributes through the vein mechanism referred to above, then these two may no longer be related and temporal response measured about the echo will be a parabola. From this, a two parameter model can be used to extract the heme from non-heme iron. Finally, as a means to accomplish this unambiguously, a contrast agent is used to modify the local susceptibility in a known way and repeat the experiment.

The above results are compared with T1, T2 and diffusion weighted imaging to touch base with previous measurements in the field. The multi-echo, spin echo sequence described in the animal model section is to measure T1. A 3D variable angle method and a conventional 2D multiple IR sequence are used to estimate T1 values.

A complete error analysis is performed for each of the four measurements: phase, R2′, R2 and R1. Discrepancies between the methods are noted. Signal recovery can occur in a number of instances which ruin the correlation with iron for R1 and R2 methods but does not affect phase or R2′.

Another simple approach to remove all linear phase effects is to use a double echo method. The phase from the first echo is used to predict the phase from the second echo based upon the simple linear dependence of phase expected from background phase effects. If the phase of the first echo, γΔBTE1, is multiplied by TE2/TE1 to predict γΔBTE2, and the predicted phase subtracted from that of the second echo (usually accomplished by complex division) then the expected phase of the corrected image will be zero. This eliminates any non-linear effects associated with a two compartment model where the phase effects are not simply additive and enables separation of small local within pixel effects.

The phase expected from heme and non-heme sources using the known estimates for the susceptibility of ferritin and its concentration of 1450 nmol/gm taken from the red nucleus is estimated using a multi-echo spatial technique to quantify the heme component. The heme iron reveals itself as an oscillatory effect for blood but not for free iron or iron in ferritin. This technique is sensitive to partial volume effects coming from venous blood and should be distinct from the effects caused by a uniform distribution of iron in the brain parenchyma. The same sequence is performed with a blood nulling technique. The amount of phase behavior that comes from heme iron (if any) versus free iron or iron bound in ferritin is quantified. The effects of blood may be determined by using a known quantity of contrast agent (the conventional agents have a phase effect of 1°/mM/ms) to mimic the susceptibility of blood. By doubling the effect of blood, the effect of blood itself on the phase is estimated. To address the issue of resolution and scale dependence, the experiments can be performed at resolutions ranging from 0.5 to 2 mm in humans and 0.25 to 1 mm in animals to determine if there is any effect of voxel size on the measurements. Any changes to the phase of the image in the parenchyma or in the critical time tₛ is a marker of the blood's contribution.

Study of the phase behavior and the ability of the phase filter to remove background material can be made. Since the filter used is a high pass filter, a loss of DC information results. As this is important to the quantification of the susceptibility (differences between tissues is not highly affected), the ability to extract the DC level phase is examined and compared with the original unfiltered data. To ensure an absolute measure of phase, not just phase differences, reference markers of known susceptibility are used. Imaging of both animals and humans is performed.

In the present invention, MR imaging is used to monitor patients. The MR imaging is sensitive to the early formation of beta-amyloid plaque and associated iron content in AD and degenerative effects in the later stages from plaque and from vascular changes and provides a means to quantify brain iron.

Another aspect of the invention concerns methods of early intervention and/or possible prevention of AD. Since subjects at risk for AD display mild cognitive impairment, including executive function and memory, for months to years prior to the aggressive and devastating expression of the disorder, longitudinal follow-up of AD cases with specific discriminating neuropsychological studies that increase diagnostic accuracy for AD, and detailed cognitive testing have proven useful.

Since mild cognitive disorder (MCI), a disorder of subthreshold dementia has been suggested to be linked with AD, a preferred embodiment of the invention concerns a target population of MCI patients. Subjects with this diagnosis perform 1.0 to 1.5 standard deviations below the reference standard for normals on standard neuropsychologic examinations and are not demented by standard tests. Guidelines and practice recommendations for the early detection of MCI in the elderly have recently been published (Jolles et al., Drugs Aging, 7(6):459-79 (1995)). Preferably, the invention involves a method of distinguishing between different cases of mild cognitive disorder (MCI). More preferably, the invention includes identification of cases of MCI that remain static from those cases of MCI that rapidly progress into dementia. Further, the invention includes a method that distinguishes between dementia syndrome and those disorders that are able to be surgically treated are developed. For example, methods for differentiating between MCI and normal pressure hydrocelphalus that can be relieved by shunting procedures are developed. Further, methods for distinguishing between fronto-temporal atrophy and multi-infarct dementia disorders from AD are developed. Another preferred embodiment of the invention involves clinical monitoring of the course of dementia in MCI patients with screening instruments that include: Mini-Mental State examination, neuropsychologic tests, with supporting data from focused cognitive instruments and information.

Another preferred embodiment involves correlation between brain disorders and iron metabolism. Preferably, levels of brain iron quantitation through MRI technologies are correlated with the clinical course of dementia in patients. Further, levels of peripheral blood IRP-2 monitored through IRP-2 assays are correlated with the clinical course of dementia in patients. Patients with irregular levels of brain iron or peripheral IRP-2 are designated prime candidates for further study and for AD intervention and possible AD prevention.

100 individuals with MCI from a variety of sources are entered into the study. 50 individuals are entered in the first year and 50 individuals are entered in the second year. A referral service that encounters 6 to 8 new MCI patients per month will be the primary source of patients. A secondary source of patients includes local neurologists and psychiatrists that will be sent notices and public service messages placed on radio and television.

Subsequent to a telephone contact and a direct referral, the subject undergoes an extensive physical and neurological assessment to include psychometric studies to assess the level of impairment. These screening studies is as described by the Quality Standards Subcommittee of the American Academy of Neurology.

The entry process consists of either a telephone interview with the study coordinator or direct referral. The first visit is an interview after selection on the basis of a mail-in questionnaire on dementia symptoms.

The guidelines of the Quality Standards Subcommittee is followed for subject selection. Criteria for entry into the study includes the following:
1) Age>50 years
2) Education more than 7^{th} grade
3) No history of a major neurological disorder: stroke, tumor, trauma, endocrinopathy, drug abuse, or psychiatric disorder.
4) No history of neurolytic drug maintenance
5) A score on the Mini-Mental State Examination ≧10
6) Able to give informed consent
7) An experienced conservator, family member

Each patient evaluation is completed in 2 weeks and consist of 3 sessions. Records are coded and standard information: psychometric scores, blood analysis results, MRI data will be abstracted into a special coding form. Data will be reviewed on a biweekly basis. All subjects give informed consent according to guidelines approved by the Human Subjects Research Committee of Loma Linda University Medical Center and the NIH.

### EXAMPLE 1

### Preparation of Antibodies Specific for IRP-2 Peptides

Antibodies specific for oxidized and reduced forms of wild type and mutant IRP-2 peptides were prepared as follows. Seven clones having one or more cysteine residues in the peptide loop of amino acid residues 138-216 of IRP-2 substituted with alanine were created by conventional techniques in molecular biology. The “C1A” clone has a substitution of the first cysteine proximal to the N-terminus with an alanine. (SEQ ID NO: 4). The “C2A” clone has a substitution of the second cysteine proximal to the N-terminus with an alanine. (SEQ ID NO: 6). The “C3A” clone has a substitution of the third cysteine proximal to the N-terminus with an alanine. (SEQ ID NO: 8). The “C12A” clone has substitutions of the first and second cysteines proximal to the N-terminus with an alanine. (SEQ ID NO: 10). The “C23A” clone has substitutions of the second and third cysteines proximal to the N-terminus with an alanine. (SEQ ID NO: 12). The “C13A” clone has substitutions of the first and third cysteines proximal to the N-terminus with an alanine. (SEQ ID NO: 14). The “CC123A” clone has substitutions of the first, second, and third cysteines proximal to the N-terminus with an alanine. (SEQ ID NO: 16). A wild type peptide sequence was also produced recombinantly in E. Coli. (SEQ ID NO: 2).

Once the recombinant peptides were isolated, they were either oxidized or reduced. Oxidation of IRP-2 was performed at the concentration of 0.1 □g/□l protein in a 20:1 reaction mixture (25 mM Hepes-NaOH, pH 7.2 and 40 mM KCl) in the presence of 50:M FeCl₃ and 10 mM DTT at 37° C. for 15-30 minutes. The reduced forms of the peptides were obtained by incubating the peptide in Tris-carboxyethyl-phosphine (TCEP) at 1 mM for 15-30 minutes at 37° C. Once the oxidized and reduced peptides were obtained, they were coupled with KLH and were used to generate antibodies in mice. Hybridomas were made using conventional methods and the clones were screened for the production of antibodies specific for the particular peptide used to inoculate the mouse. The antibody generated to the wild type peptide was found to recognize both the peptide of SEQ. ID. NO: 2 and full-length IRP-2 in both ELISA and Western blot. A 1:5000 dilution was found sufficient. Another selection process was also used to screen some of the antibodies. Because the oxidation of IRP-2 can depend on the conversion of a cysteine residue to aminomalonic acid, an IRP-2 peptide having aminomalonic acid was synthesized. The clones were screened for reactivity to the aminomalonic acid peptide and also the native IRP-2 peptide. Clones that were reactive to the aminomalonic peptide but not the native peptide were selected. By using the teachings described in this example, antibodies to both mutant and wildtype IRP-2 proteins can be made. These antibodies can be used in the diagnostic assays described herein to identify a subject's predilection to a neurodegenerative disease. The next example describes a similar approach that was used to make an antibody specific for wild-type IRP-2.

### EXAMPLE 2

### Antibodies Specific for IRP-2

In this example, an approach that was used to make and screen an antibody specific for IRP-2 is provided. To make the antibody, Balb/c mice were immunized with a 63-residue (wild type) “loop peptide”, in RIBI Adjuvant (Corixa) following the manufacturers protocol. Splenocytes from the mice were then fused to Sp2/0 myeloma cells using standard hybridoma techniques. The resulting hybridomas were screened for reactivity with the loop peptide, as well as the whole molecule. Six clones were positive by ELISA (native molecule) and Western Blot (denatured molecule). One of these (4G11) was selected for large scale antibody production, based on it's growth characteristics and strong assay results. This hybridoma was deposited under the terms of the Budapest Treaty with the American Type Culture Collection, 10801 University Blvd., Manassas, Va. 20110-2209, USA (ATCC) as deposit number on the date

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, which assures that all restrictions imposed by the depositor on the availability to the public of the deposited material will be irrevocably removed upon the granting of the pertinent U.S. patent, assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC '§122 and the Commissioner's rules pursuant thereto (including 37 CFR §1.14 with particular reference to 886 OG 638).

Competitive binding assays were then conducted between 4G11 and the other 5 clones, to determine whether they recognized the same or different epitopes. Only one (14F7) did not significantly inhibit the binding of 4G11, and is assumed to bind at a different site. Thus, 14F7 and 4G11-HRP became the basis for the capture ELISA assay (described below), which can also be used for detection of IRP-2 in biological samples. The assay is sensitive down to 1 □g/mL and shows excellent linearity.

The capture assay was performed as follows. Unlabeled antibody was diluted in carbonate buffer, pH 9.6 (Sigma #C-3041), usually to 1-10 ug/mL. The individual antibody concentration may need to be determined empirically, starting with 10 ug/mL and working downward. It is important not hinder antigen binding by overcrowding and the lowest concentration that will still give a strong signal was selected. The antibodies were then plated, approx. 100 □L per well, in IMMULON-1 plates (Dynex #3355), covered with tape (Falcon #3073), and incubated overnight at 4° C.

Subsequently, the plates were warmed to room temp. and the wells were washed 3× with PBS (w/o tween) (Cellgro, #20-031-CV, 10× Concentrate diluted to 1×.) The plates were then blocked with SuperBlock (Pierce #37515), by adding 200:L to each well, emptying by inversion, and repeating the process for a total of 3×. The wells were then washed 3× with PBS-Tween (PBS +0.05% Tween-20, Sigma #P-6585). Diluent (control), antigen, and standards (approx. 100 □l) were added to the wells. The diluent used was carrier (10% SuperBlock in PBS-Tween). The wells were taped and the reaction was allowed to take place for 1 hr at room temp. (Shaking the plate will greatly increase sensitivity in assays). Subsequently, the wells were washed 3× with PBS-Tween.

Next, approx. 100 □L of an HRP-tagged detection antibody diluted in carrier was added. (The manufacturer's recommendations was followed when a commercial product was used and the appropriate dilution of home grown antibodies was empirically determined.) The wells were taped and the reaction was allowed to take place for 1 hr at room temp. (Shaking the plate will greatly increase sensitivity in assays). Subsequently, the wells were washed 3× with PBS-Tween. Then approx. 100 uL substrate (Bio-Rad #172-1067) was added to the wells. The reaction was allowed to take place uncovered for approx. 30 min. and readings at an absorbance at 630 nm, ref. 490 nm were taken. Assay parameters were such that an O.D. of around 2.0 was obtained in about 30 min. incubation for the most concentrated antigen samples in the assay. When scouting out appropriate dilutions of capture antibody, antigen, and detection antibody, it may be helpful to do a “checkerboard” assay. The example below describes an approach that was used to join antiIRP-2 antibodies to beads.

### EXAMPLE 3

### Support-Bound IRP-2 Antibodies

This example describes an approach that was used to prepare support-bound IRP-2 antibodies for use in flow cytometry. Approximately, five milligrams of purified, carrier-free (no other proteins), mouse monoclonal antibody directed against IRP-2 was modified with sulfo-SMCC and then was conjugated to 15 mg of r-phycoerythrin modified with 2-iminothiolane. The resultant conjugate was separated from free unconjugated r-phycoerythrin and free unconjugated monoclonal antibody by size exclusion chromatography on Sepharose S-300-HR columns. The procedure required two days to complete. Final yield of usable conjugate was about 50-95% of initial antibody mass with usual anticipated yields of >85%. Successful conjugation was confirmed by capture of conjugate on goat anti-mouse coated 7 micron beads and analysis by flow cytometry.

### EXAMPLE 4

### Preparation of Leukocytes

Mononuclear cells are prepared from heparin anticoagulated peripheral blood samples by density gradient separations using 68% Percoll. Briefly, 20 ml of undiluted whole blood samples are layered onto 25 ml of 68% Percoll in a 50 ml centrifuge tube. The blood is then centrifuged for 20 minutes at 800×g. The interface cells are collected and pelleted by centrifugation The cell pellet is disrupted by vortexing and the remnant erythrocytes are removed by lysis using 25 ml of VitaLyse erythrocyte lysing buffer (BioErgonomics). Cells are washed once with 25 ml of PBS and then resuspended to 1×10⁷ mononuclear cells/ml. One hundred microliters of cells (1×10⁶) are used for each labeling procedure.

For stimulation of inflammatory cytokine expression, cells are resuspended in either Basal Medium or ActiCyte-LPS medium (BioErgonomics, St. Paul, Minn.) at a concentration of 1×10⁶ cells/ml and incubated for 20 hours at 37° C. in an atmosphere of 5% CO₂. During the last 4 hours of incubation, the golgi inhibitor Brefeldin A (10 ng/ml) is added to the cultures to inhibit the secretion of cytokine and enhance the intracellular staining. After the incubation period, cells are harvested and the culture supernatants retained for cytokine secretion analysis. Cells are retained for detection of intracellular cytokines.

Activation of lymphocytes for the detection of altered levels of intracellular IRP-2 protein or induction of apoptosis are performed in the following manner. One million mononuclear cells are resuspended in either Basal Medium or ActiCyte-TC medium (BioErgonomics, St. Paul, Minn.) and incubated for 48-72 hours at 37° C. in an atmosphere of 5% CO₂. ActiCyte-TC medium contains anti-CD3 antibody and the human cytokines Interleukin-1 alpha (IL-1α) and Interleukin-2 (IL-2). This medium specifically activates T-lymphocytes via the epsilon chain of the T-cell antigen receptor and the receptors for the two cytokines.

### EXAMPLE 5

### Fluorochrome-Labeled Anti-IRP-2 Antibodies

1. Fluorochrome Labeling of Antibodies

To label antibodies with FITC, antibodies are exchanged into 100 mM KH₂CO₃ buffer (pH 9.0) at a concentration of 5 mg/ml. FITC (Molecular Probes) (10 mg/ml in DMF) is added to the antibody at a 25:1 molar ration and incubated for 1 hour at room temperature, in the dark. Free FITC is separated from the antibody on a G-25 Sephadex column. Phycoerythrin and Cy5PE conjugates are produced using 2-iminothiolane to modify the fluorochrome and sulfo-SMCC to modify the antibody. The modified proteins are then incubated together for 1 hour at room temperature in the dark. Free fluorochrome and antibody is separated from fluorochrome-conjugated antibody by separation on Sephacryl S-300-HR columns (Sigma). Alterations in the ratio of fluorochrome to protein may be necessary to optimize the fluorescent signal for a particular antibody or peptide antigen.

2. Quality Control of Anti-IRP-2 Antibodies and Fluorochrome-Labeled Anti-IRP-2 Antibodies

The antibodies that are developed against the IRP-2 native, mutant peptides and intact proteins are tested for specificity using both antigen-down ELISA and a micro particle-based immunofluorescent assay developed at BioErgonomics, Inc. Biotin-labeled native and mutant peptides or intact IRP-2 proteins are attached to 7 μm diameter avidin-coated polystyrene paramagnetic particles that bind, with high specificity and avidity, biotin-labeled molecules. The newly developed antibodies are tested for specificity against the micro particles coated with the individual various IRP-2 peptides by sandwich assay. IRP-2-specific antibody bound to the antigen-coated particles is detected by subsequent reaction with phycoerythrin-labeled goat anti-mouse Ig antibody. Samples are analyzed by flow cytometry. Antibodies that produce a positive fluorescence signal are considered potentially specific for the native or mutant peptides. Specificity is confirmed by blockade of specific binding and fluorescence of anti-IRP-2 antibody by pre-incubation of cells or antigen-coated particles with the same unlabeled antibody or pre-incubation of labeled antibody with antigen prior to incubation with the cell or antigen-coated micro particle.

Antigen-coated microparticles are used for quality control of the fluorescent conjugation of the previously selected IRP-2 specific antibodies. Optimal labeling of the anti-IRP-2 antibodies with either phycoerythrin or Cy5-phycoerythrin fluorescent dyes which produce optimal signal-to-noise ratios are selected based on binding to antigen-coated micro particles and intracellular labeling of both antigen-positive and antigen-negative cells populations. Grouping of specificities of antibodies for particular epitopes of the IRP-2 peptides or complete molecules are determined by specific blockade of fluorochrome-labeled antibodies with unlabeled antibodies.

### EXAMPLE 6

### Analysis of Peripheral Blood Samples From MCI Patients

Peripheral blood samples will be obtained from MCI patients. Blood assays are performed twice per year at a minimum.

1. Assay for Expression of Surface Membrane Forms of βAPP

The relative expression of cell surface membrane forms of βAPP are determined for study subjects by flow cytometric analysis. Briefly, isolated mononuclear cells are stained with the monoclonal antibody 22C11 which is specific for the n-terminus of the βAPP (Boehringer Mannheim) for 30 minutes and phycoerythrin-conjugated CD14. After incubation with the antibodies, cells are washed once with PBS to remove unbound antibody and the cells are then analyzed by flow cytometry.

2. Assay for Expression of Functional Cell Surface Transferrin Receptors

The relative expression of functional transferrin receptors on cells from test subjects is determined by flow cytometric analysis. Briefly, isolated mononuclear cells are stained with 100 ng of phycoerythrin-conjugated human transferrin (BioE Inc.) for 15 minutes and washed once with PBS to remove unbound conjugate prior to flow cytometric analysis. Expression of functional receptors (that is, receptors actually capable of binding transferrin) is directly proportional to the intensity of fluorescence of the cells.

3. Assay for Expression of Proinflammatory Cytokines by Circulating Leukocytes

Mononuclear cells (1×10⁶/ml) are incubated for 20 hours in the presence or absence of bacterial lipopolysaccharide (LPS) to determine basal and stimulated production of the proinflammatory cytokines Interleukin-1 alpha (IL-1α), Interleukin-6 (IL-6), and Tumor Necrosis Factor-alpha (TNF-α). Identification of cytokine producing cells is performed by flow cytometry by analysis of intracellular cytokines. Briefly, cells are fixed and permeabilized as described for the identification of IRP-2 proteins. The cells are labeled with PE- or Cy5PE-labeled antibodies specific for IL-1 α, IL-6 and TNF-α. The amount of cytokine secreted into the culture media during the 20 hour incubation is measured by a flow cytometric-based quantitative immunofluorescent assay (ImmunoFlow and MultiFlow, BioErgonomics, Inc., St Paul, Minn.).

4. Assay for Detection of Apoptosis or Necrosis in Activated Mononuclear Cells

Briefly, one million mononuclear cells activated by ACTICYTE-TC are washed with PBS and stained with the phosphatidylserine binding protein Annexin-V-FITC (200 ng, Caltag, South San Francisco, Calif.) and the DNA-intercalating dye Propidium Iodide (4 ug). Cells that are positive for Annexin-V alone or Annexin-V and Propidium Iodide are considered as early or late-stage apoptotic, respectively, while cells that are positive for Propidium iodide alone are considered necrotic.

5. Assay for Expression of Intracellular IRP-2 Loop Peptides and Identification of IRP-2 Expressing Cells

Expression of intracellular IRP-2 loop peptide is determined by flow cytometric analysis using FITC, PE and Cy5PE-conjugated anti-IRP-2 monoclonal antibodies to identify cells expressing the native IRP-2 proteins. Experiments are projected to search for IRP-2 iron degradation domain polymorphism. Washed cells (1×10⁶ in 100 μl of PBS) are fixed by incubation in 1 ml of 1% formaldehyde for 30 minutes expression by specific binding of fluorescently-labeled antibodies directed against intracellular IRP-2 proteins. Positive fluorescence and identification of specificity for a particular anti-IRP-2 antibody is determined by a shift in fluorescence intensity that can be specifically competed by preincubation with antigen or unlabeled antibody. Cells positive for a particular anti-CD antibody is determined by a comparison to similarly-labeled isotypic control antibody or cells whose fluorescent staining was specifically-blocked by unlabeled antibody.

6. Correlation of Flow Cytometric Data and Patient's Clinical Status

The flow cytometric data is correlated with the clinical status of the patient in a separate filing system with a data sheet for each patient. The results of the IRP-2 “loop peptide” degradation screen is analyzed in the MCI subject group using analysis of variance (ANOVA). Data comparing mutated IRP-2 peptide in the two population cohorts (AD and elderly control) may further be examined. Although the invention has been described with reference to embodiments and examples, it should be understood that various modifications can be made without departing from the spirit of the invention. Accordingly, the invention is limited only by the following claims. All references cited herein are hereby expressly incorporated by reference.

What is claimed is:

1. A method of identifying a subject as likely to develop or have Alzheimer's disease or Mild Cognitive Impairment (MCI), comprising:
obtaining a biological sample having peripheral blood cells from said subject, said sample having protein;
providing a probe that interacts with a wild type iron-regulatory protein-2 (IRP-2) (SEQ ID NO:18) and/or mutant IRP-2, wherein said mutant IRP-2 comprises one or more mutations in SEQ ID NO:18;
contacting the biological sample with the probe under conditions that allow the probe to interact with wild-type and/or mutant IRP-2 protein that is present in the biological sample;
measuring the amount of probe detected after contacting the sample with the probe; and
identifying the subject as likely to develop or have Alzheimer's disease or MCI, by detecting significantly more probe that interacts with wild-type and mutant IRP-2 protein in the biological sample than would be detected in a control sample.

2. The method of claim 1, wherein the probe is selected from the group consisting of a nucleic acid, a protein, and a peptidomimetic.

3. The method of claim 1, wherein the detection of the amount of probe that interacts with the protein comprises use of a technique selected from the group consisting of fluorescence-activated cell sorting (FACs), immunoprecipitation, Western blot, immunochromatography, antibody staining, and a hybridization assay.

4. A method for the identification of a defect in iron metabolism in a patient, comprising:
obtaining a biological sample having peripheral blood cells from said subject, said sample having protein;
providing a probe-that interacts with a wild type iron regulatory protein 2 (IRP-2) (SEQ ID NO:18) and/or mutant IRP-2 protein, wherein said mutant IRP-2 comprises one or more mutations in SEQ ID NO:18;
contacting the biological sample with the probe under conditions that allow the probe to interact with the wildtype and/or mutant IRP-2 protein that is present in the biological sample;
measuring the amount of probe detected after contacting the sample with the probe; and
identifying the subject as having a defect in iron metabolism by detecting significantly less or more probe that interacts with the wildtype and/or mutant IRP-2 protein in the biological sample than would be detected in a control sample.


SEQUENCE LISTING (20 sequences)

SEQ ID NO: 1 - DNA, 189 bp, Artificial Sequence
  cloning oligonucleotide
gcaatacagaatgcaccaaatcctggaggtggtgacctgcagaaagcaggaaagctctctccacttaaagtgcagcctaagaagcttccctgcagaggccagactacctgccgaggatcttgtgattctggagaactaggccgaaactcaggaacattttcttcgcagattgagaatacacccatcctg

SEQ ID NO: 2 - protein, 63 aa, Artificial Sequence
  peptide for antibody production
AIQNAPNPGGGDLQKAGKLSPLKVQPKKLPCRGQTTCRGSCDSGELGRNSGTFSSQIENTPIL

SEQ ID NO: 3 - DNA, 189 bp, Artificial Sequence
  cloning oligonucleotide
gcaatacagaatgcaccaaatcctggaggtggtgacctgcagaaagcaggaaagctctctccacttaaagtgcagcctaagaagcttcccgccagaggccagactacctgccgaggatcttgtgattctggagaactaggccgaaactcaggaacattttcttcgcagattgagaatacacccatcctg

SEQ ID NO: 4 - protein, 63 aa, Artificial Sequence; identical to SEQ ID NO: 2
  peptide for antibody production

SEQ ID NO: 5 - DNA, 189 bp, Artificial Sequence
  cloning oligonucleotide
gcaatacagaatgcaccaaatcctggaggtggtgacctgcagaaagcaggaaagctctctccacttaaagtgcagcctaagaagcttccctgcagaggccagactaccgcccgaggatcttgtgattctggagaactaggccgaaactcaggaacattttcttcgcagattgagaatacacccatcctg

SEQ ID NO: 6 - protein, 63 aa, Artificial Sequence
  peptide for antibody production
AIQNAPNPGGGDLQKAGKLSPLKVQPKKLPCRGQTTARGSCDSGELGRNSGTFSSQIENTPIL

SEQ ID NO: 7 - DNA, 189 bp, Artificial Sequence
  cloning oligonucleotide
gcaatacagaatgcaccaaatcctggaggtggtgacctgcagaaagcaggaaagctctctccacttaaagtgcagcctaagaagcttccctgcagaggccagactacctgccgaggatctgctgattctggagaactaggccgaaactcaggaacattttcttcgcagattgagaatacacccatcctg

SEQ ID NO: 8 - protein, 63 aa, Artificial Sequence
  peptide for antibody production
AIQNAPNPGGGDLQKAGKLSPLKVQPKKLPCRGQTTCRGSADSGELGRNSGTFSSQIENTPIL

SEQ ID NO: 9 - DNA, 189 bp, Artificial Sequence
  cloning oligonucleotide
gcaatacagaatgcaccaaatcctggaggtggtgacctgcagaaagcaggaaagctctctccacttaaagtgcagcctaagaagcttcccgccagaggccagactaccgcccgaggatcttgtgattctggagaactaggccgaaactcaggaacattttcttcgcagattgagaatacacccatcctg

SEQ ID NO: 10 - protein, 63 aa, Artificial Sequence
  peptide for antibody production
AIQNAPNPGGGDLQKAGKLSPLKVQPKKLPARGQTTARGSCDSGELGRNSGTFSSQIENTPIL

SEQ ID NO: 11 - DNA, 189 bp, Artificial Sequence
  cloning oligonucleotide
gcaatacagaatgcaccaaatcctggaggtggtgacctgcagaaagcaggaaagctctctccacttaaagtgcagcctaagaagcttccctgcagaggccagactaccgcccgaggatctgctgattctggagaactaggccgaaactcaggaacattttcttcgcagattgagaatacacccatcctg

SEQ ID NO: 12 - protein, 63 aa, Artificial Sequence
  peptide for antibody production
AIQNAPNPGGGDLQKAGKLSPLKVQPKKLPCRGQTTARGSADSGELGRNSGTFSSQIENTPIL

SEQ ID NO: 13 - DNA, 189 bp, Artificial Sequence
  cloning oligonucleotide
gcaatacagaatgcaccaaatcctggaggtggtgacctgcagaaagcaggaaagctctctccacttaaagtgcagcctaagaagcttcccgccagaggccagactacctgccgaggatctgctgattctggagaactaggccgaaactcaggaacattttcttcgcagattgagaatacacccatcctg

SEQ ID NO: 14 - protein, 63 aa, Artificial Sequence
  peptide for antibody production
AIQNAPNPGGGDLQKAGKLSPLKVQPKKLPARGQTTCRGSADSGELGRNSGTFSSQIENTPIL

SEQ ID NO: 15 - DNA, 189 bp, Artificial Sequence
  cloning oligonucleotide
gcaatacagaatgcaccaaatcctggaggtggtgacctgcagaaagcaggaaagctctctccacttaaagtgcagcctaagaagcttcccgccagaggccagactaccgcccgaggatctgctgattctggagaactaggccgaaactcaggaacattttcttcgcagattgagaatacacccatcctg

SEQ ID NO: 16 - protein, 63 aa, Artificial Sequence
  peptide for antibody production
AIQNAPNPGGGDLQKAGKLSPLKVQPKKLPARGQTTARGSADSGELGRNSGTFSSQIENTPIL

SEQ ID NO: 17 - DNA, 2867 bp, Homo Sapiens
taccttattgaaacattaaatgacagttcacataagaagttcttcgatgtatctaaacttggcaccaagtatgatgttctgccttactcaatacgggtcttgttggaagctgctgtacgaaattgtgatggctttttaatgaagaaggaagatgttatgaacattttagactggaaaaccaaacaaagcaatgttgaagtgccctttttccctgcccgtgttcttcttcaagattttactggaataccagcaatggtggattttgctgctatgagggaggcagtgaaaactcttggaggtgatcctgagaaagtccatcctgcttgtccgacagatcttacagttgaccattctttacaaattgacttcagtaaatgtgcaatacagaatgcaccaaatcctggaggtggtgacctgcagaaagcaggaaagctctctccacttaaagtgcagcctaagaagcttccctgcagaggccagactacctgccgaggatcttgtgattctggagaactaggccgaaactcaggaacattttcttcgcagattgagaatacacccatcctgtgtccttttcatttgcaaccagtgcctgaacctgaaacagtgttaaaaaatcaagaagtagaattcggcagaaatcgagagaggcttcagttttttaagtggagttcaagagttttaaagaatgtggcagtgatccctcctggaactggaatggctcatcaaataaacttagaatatttgtcaagagtggtttttgaagaaaaagacctcctcttcccagacagtgtagtcggcacagattcacacataacgatggtgaatggtttagggattctggggtggggggttggaggcattgaaacagaagcagttatgcttggtctgccagtttctcttactttaccagaggtggttggatgtgagttaactgggtcatcaaacccttttgttacatccatagatgttgttcttggtattacaaagcacctcaggcaagtaggagtggctggaaagtttgttgagttttttggaagtggagtttcacaattatctatagttgatcgaactacaatagcaaacatgtgtccggaatatggtgctatcctcagctttttccctgttgacaatgtgacattaaaacatttagaacatacaggttttagcaaagccaaactcgaatcaatggaaacataccttaaagctgtgaaattgtttcgaaatgaccagaattcttcaggagaacctgaatactcccaggtgatccagattaatctgaattcaatagttccatctgttagtggtccaaaaagacctcgggatagagttgctgtgacagatatgaaaagcgatttccaggcttgcttaaatgaaaaggttggatttaaaggcttccaaattgcagctgaaaaacaaaaggatattgtctccattcattatgaaggaagtgaatataagctgtctcatggatcagtggtcattgctgcagttatcagttgtaccaataattgcaatccatctgtcatgcttgctgcaggtcttttggctaaaaaggctgttgaagctggtctgcgtgttaaaccttatataagaacaagtttatctccaggcagtgggatggttacacattacctcagttcaagtggagtattaccatatctaagtaagcttggatttgaaatcgttggctatggatgttcaacttgtgtgggaaatacagcacccttatcagacgcagttttaaatgcagtaaaacagggtgatttggttacctgtggtaattttatctggaaaaaaaattttgaaggtcgtctttgtgattgtgttcgtgccaattatcttgcctctccacccttagtggtagcttatgccatagcaggcacagtgaatatagatttccagacagaacctttaggtactgaccccaccggcaagaacatttacctgcatgatatttggcctagtcgagaagaagttcatcgagtagaggaagaacatgttatactatccatgtttaaagcattaaaagataaaatagaaatggggaataaacggtggaattccttagaagcaccggattcagttttgtttccatgggacttaaagtctacttatatcagatgcccttcattttttgataaacttaccaaagagccaattgcactccaggctattgaaaatgcccatgtcttattatatttgggagactctgtcacaacagatcatatatcacctgcaggaagtatcgctaggaatagtgctgccgctaagtatttgacaaacagaggccttacccctcgtgaattcaactcttacggagctcgaagaggtaatgatgctgtaatgacaagaggcacttttgcaaatatcaagctttttaataagtttattggaaaaccagctcctaaaacaattcattttccatcaggacagacgctagatgtatttgaggctgcagagctgtaccagaaagaaggtatcccactgattattttagcaggaaagaaatatggttcaggaaactccagagactgggctgccaaaggaccgtatttactgggtgtgaaagctgttttggccgaaagttatgaaaaaatacacaaagatcatttgattggaattggcatagctccacttcagttccttccaggagaaaatgcagattccttgggcctctccggtagagaaacattttctttaacatttcctgaagaactgtctcctggaattacattgaatatacagacaagcactggaaaagtattcagcgtgattgcttcgtttgaagatgatgtggaaataacattatacaaacatggaggattattaaactttgtggcacgaaaattctcatagtatctact

SEQ ID NO: 18 - protein, 952 aa, Homo Sapiens
YLIETLNDSSHKKFFDVSKLGTKYDVLPYSIRVLLEAAVRNCDGFLMKKEDVMNILDWKTKQSNVEVPFFPARVLLQDFTGIPAMVDFAAMREAVKTLGGDPEKVHPACPTDLTVDHSLQIDFSKCAIQNAPNPGGGDLQKAGKLSPLKVQPKKLPCRGQTTCRGSCDSGELGRNSGTFSSQIENTPILCPFHLQPVPEPETVLKNQEVEFGRNRERLQFFKWSSRVLKNVAVIPPGTGMAHQINLEYLSRVVFEEKDLLFPDSVVGTDSHITMVNGLGILGWGVGGIETEAVMLGLPVSLTLPEVVGCELTGSSNPFVTSIDVVLGITKHLRQVGVAGKFVEFFGSGVSQLSIVDRTTIANMCPEYGAILSFFPVDNVTLKHLEHTGFSKAKLESMETYLKAVKLFRNDQNSSGEPEYSQVIQINLNSIVPSVSGPKRPRDRVAVTDMKSDFQACLNEKVGFKGFQIAAEKQKDIVSIHYEGSEYKLSHGSVVIAAVISCTNNCNPSVMLAAGLLAKKAVEAGLRVKPYIRTSLSPGSGMVTHYLSSSGVLPYLSKLGFEIVGYGCSTCVGNTAPLSDAVLNAVKQGDLVTCGNFIWKKNFEGRLCDCVRANYLASPPLVVAYAIAGTVNIDFQTEPLGTDPTGKNIYLHDIWPSREEVHRVEEEHVILSMFKALKDKIEMGNKRWNSLEAPDSVLFPWDLKSTYIRCPSFFDKLTKEPIALQAIENAHVLLYLGDSVTTDHISPAGSIARNSAAAKYLTNRGLTPREFNSYGARRGNDAVMTRGTFANIKLFNKFIGKPAPKTIHFPSGQTLDVFEAAELYQKEGIPLIILAGKKYGSGNSRDWAAKGPYLLGVKAVLAESYEKIHKDHLIGIGIAPLQFLPGENADSLGLSGRETFSLTFPEELSPGITLNIQTSTGKVFSVIASFEDDVEITLYKHGGLLNFVARKFS

SEQ ID NO: 19 - DNA, 3770 bp, Rattus Norvegicus
tgaggccggcgatggactccccaagtgcaggatacacctttgagtaccttattgaaacattaaatggcagttcacagaagaagttcttcaatgtacctaaacttggaggcaccaagtatgatattctgccttactcaatacgggtcctattggaagctgctgtacgaaattgtgatggatttttaatgaaaaaggaagatgttataaatattttggactggaaaaccaaacaaagcaatgttgaagtgccctttttccccgcccgtgttgttcttcaagatttcactggaataccggcaatggtggattttgctgctatgagggaggcaatgaaaactcttggaggtgatcctaagaaagtccaccctgcctgtccaacagatctcacagttgaccactctttacagattgacttcagtaaatgtgcaatacagaatgcaccaaatcctggaggtggtgacctacagaaagcaggaaagctctctccacttaaagtacagcctaagaagcttccatgtcgaggccagactacctgccggggatcgtgtgattctggagaactaagccgaaactcaggaacattttcttcgcagattgagaatacgcctgtcctgtgtccctttcatttgcaaccagtgcctgaacctgagacggtgttaaaaaatcaagaagtagaatttggcagaaatcgagagaggcttcaatttttcaagtggagctcaggagcttttaagaatgtggcagtcatccctcctggaactggaatggctcatcaagtgaacttagaacatttgtcgagagtagtgtttgaagaagccgacctgctcttcccagacagcgtaattggcacagattctcatataaccatggtgaatggattgggaattcttgggtggggagttggaggcattgagacagaggcagttatgcttggcctgccagttactcttactttaccagaggtggttggatgtgagctaactgggtcatccaatgcttttgttacatccatagatattgtcctaggcattacaaagcacctcaggcaagtaggcgtggctggaaagtttgttgagttctttggaagtggagtttcacaattatctattgtcgatagaactactatagcaaacatgtgtcccgagtatggtgctatcctcagctttttccctgttgacaatgtgacactacgacatttagaacatacaggttttgacaaaaccaaacttgagtcaatggaagaataccttaaagctgtgaaattgtttcgaaatgatgagaattcttcagaacctgaatattctcaggtgatacagattaatctgaattcaatagttgcatctgtcagtggtccaaaaaggcctcaggatagagttgctgtaacagatatgaaaagtgattttcaggcttgcttaaatgaaaaggttggatttaaaggcttccaagttgcagcagaaaaacaaagtgatactgtctcagttcgttatgatggaagtgagtataagctgtcccatgggtccgtggtcattgctgcggttatcagctgtaccaataactgcaatccatcagtgatgctcgctgcaggtcttttggctaaaaaggctgttgaaactggtctacgagttaaaccttatataagaacaagtttgtctccaggcagtgggatggttacacattacctcagttcaagtggagtgttaccctaccttagcaagctagggtttgaaatagttggctatggatgttcaacgtgtgtaggaaatacagcacctttatcagaagcaattttgaatgcagtaaaacagggtgatttggctacctgtggagttttatctggaaacaaaaatttcgaaggtcgtctttgtgattgtgtccgtgctaattatctcgcctctccacccttagtggtggcttacgccatagcaggcacagtgaatatagatttccagacagagccattaggtactgactctacaggcaagaacatttacctgcatgacatttggcctagtcgagaagaagttcatcagatagaagaagaacatgttattttgtccatgtttaaagcactgaaagagaaagtagagatgggaaataaacggtggaattccttagacgctccagattcagtgttgtttccatgggatgttaagtctacttacatcagatgcccttcattttttgataaacttaccaaagaaccagctgcatcccagcctattgaaaatgcccatgtcctgttgtacttgggagactctgtcactacagaccacatatcacctgctggaagcattgctaggagcagagctgctgctaagtacttgacaaacagaggccttactccccgagagttcaactcttatggagctcgaagaggtaatgatgctgtgatgacaagaggcacgtttgcaaacatcaagctttttaataagtttattgggaagccagctcccaaaacaattcattttccatcaggacagacgctcgatgtatttgaagctgcagaattataccaaaaagaaggtatcccactgataattttagcaggaaaaaaatatggttcaggaaattcaagagactgggctgcgaaaggaccctatttgctgggtgtaaaggctgttttggctgaaagctatgaaaagatacacaaagatcatttgattggaattggtatagcaccacttgagttcctcccaggagaaaatgcagattccttgggcctctccggcagagaagtattttctttatcatttcctgaagaactctttcctggaattacgttaaatataaagacgagcactggcaaagagttcagcgtgattgcagcatttgaaaatgatgtggagataactttgtacaaacatggaggattgttaaactttgtggctcgaaaattcttatagtatctactccccatggtatctttcatggctggtaactgcaaagccttcttctgtgctggacccaggaatcattgccatggaactgcagatgatttcagtatacttctctcctccatggatgtaaatgataacgaatcaacgtagtgactaaaatgaaatcttgattttaaataatatacgaatggtgctattaacattgctaaaatcaacgtgtgaaggtgtgttgtggaagagacctgtaagtatggggggtatattttatgagaacattttgtaaataaagacagaatttgaacttgtgttgaagattcatatgaatagccgttctaaagctgtttgttttgttttgcaccttaaaactggactactgtttgttggtttaagaatagcaagttgatttagaagaagccagactagatcctaaaattatggaaatgggtacctgatttagaaatgaatttttaaatgtttttctttccagaattgaattggacacaattggcatttccagtttgtaatgtaagtcaggtttggccttagtctcaatacatctgcaaggcatagaacctgccccagatcacagtccctctgaccagccaaaatgtcctccatgtctgcagataaatgactgtaaaatacagctgattgtgtggtaccgtgtattagtaagaatatatttcctgtggtatagccctgtaattttttcagtaacttgccactatgacccactcacaagcccaaatacggtgtattaatttggactagattttgctcattttacatgactgtaactcctgtaacctcaattaggaatcactagctgacattccacactccttttatgactgctggaaacaattgagtcaa

SEQ ID NO: 20 - protein, 963 aa, Rattus Norvegicus
MDSPSAGYTFEYLIETLNGSSQKKFFNVPKLGGTKYDILPYSIRVLLEAAVRNCDGFLMKKEDVINILDWKTKQSNVEVPFFPARVVLQDFTGIPAMVDFAAMREAMKTLGGDPKKVHPACPTDLTVDHSLQIDFSKCAIQNAPNPGGGDLQKAGKLSPLKVQPKKLPCRGQTTCRGSCDSGELSRNSGTFSSQIENTPVLCPFHLQPVPEPETVLKNQEVEFGRNRERLQFFKWSSGAFKNVAVIPPGTGMAHQVNLEHLSRVVFEEADLLFPDSVIGTDSHITMVNGLGILGWGVGGIETEAVMLGLPVTLTLPEVVGCELTGSSNAFVTSIDIVLGITKHLRQVGVAGKFVEFFGSGVSQLSIVDRTTIANMCPEYGAILSFFPVDNVTLRHLEHTGFDKTKLESMEEYLKAVKLFRNDENSSEPEYSQVIQINLNSIVASVSGPKRPQDRVAVTDMKSDFQACLNEKVGFKGFQVAAEKQSDTVSVRYDGSEYKLSHGSVVIAAVISCTNNCNPSVMLAAGLLAKKAVETGLRVKPYIRTSLSPGSGMVTHYLSSSGVLPYLSKLGFEIVGYGCSTCVGNTAPLSEAILNAVKQGDLATCGVLSGNKNFEGRLCDCVRANYLASPPLVVAYAIAGTVNIDFQTEPLGTDSTGKNIYLHDIWPSREEVHQIEEEHVILSMFKALKEKVEMGNKRWNSLDAPDSVLFPWDVKSTYIRCPSFFDKLTKEPAASQPIENAHVLLYLGDSVTTDHISPAGSIARSRAAAKYLTNRGLTPREFNSYGARRGNDAVMTRGTFANIKLFNKFIGKPAPKTIHFPSGQTLDVFEAAELYQKEGIPLIILAGKKYGSGNSRDWAAKGPYLLGVKAVLAESYEKIHKDHLIGIGIAPLEFLPGENADSLGLSGREVFSLSFPEELFPGITLNIKTSTGKEFSVIAAFENDVEITLYKHGGLLNFVARKFL